(19) United States
(12) Patent Application Publication
(10) Pub. No.: US 2015/0250769 A1
(43) Pub. Date: Sep. 10, 2015

(54) METHODS AND COMPOSITIONS FOR INHIBITING RHO/MRTF-MEDIATED DISEASES AND CONDITIONS

(71) Applicants: THE REGENTS OF THE UNIVERSITY OF MICHIGAN, Ann Arbor, MI (US); BOARD OF TRUSTEES OF MICHIGAN STATE UNIVERSITY, East Lansing, MI (US)

(72) Inventors: Scott D. Larsen, South Lyon, MI (US); Jessica L. Bell, Ann Arbor, MI (US); Richard Neubig, Ann Arbor, MI (US); Kim Hutchings, Dexter, MI (US)

(21) Appl. No.: 14/642,203

(22) Filed: Mar. 9, 2015

Related U.S. Application Data
(60) Provisional application No. 61/950,373, filed on Mar. 10, 2014.

(51) Int. Cl. A61K 31/4245; A61K 45/06

(52) U.S. Cl. CPC A61K 31/4245; A61K 45/06

(57) ABSTRACT

The present disclosure relates to methods, compositions, and kits for the inhibition of signaling by members of the Rho GTPase family. Specifically, the disclosure relates to methods, compositions and kits for the inhibition of RhoA and/or RhoC transcriptional signaling and action of the transcription co-factors MRTF-A and/or MRTF-B. The disclosure finds use in treatment of Rho-mediated disease states (e.g., tumor metastasis and fibrosis), Rho-mediated biological conditions, and in cell signaling research.

The present application claims priority to U.S. Provisional Patent Application Ser. No. 61/950,373, filed Mar. 10, 2014, the disclosure of which is herein incorporated by reference in its entirety.

### FIELD OF THE DISCLOSURE

The present disclosure relates to methods, compositions, and kits for the inhibition of signaling by members of the Rho GTPase family. Specifically, the disclosure relates to methods, compositions and kits for the inhibition of RhoA and/or RhoC transcriptional signaling and action of the transcription co-factors MRTF-A and/or MRTF-B. The disclosure finds use in treatment of Rho-mediated disease states (e.g., tumor metastasis and fibrosis), Rho-mediated biological conditions, and in cell signaling research.

### BACKGROUND OF THE DISCLOSURE

Cancer metastasis is a significant medical problem in the United States, where it is estimated that >500,000 cancer-related deaths in 2003 resulted from metastatic tumors rather than primary tumors (approximately 90% of cancer deaths). Cancer metastasis requires malfunction in several tightly regulated cellular processes controlling cell movement from a primary site to a secondary site. These cellular processes include cell survival, adhesion, migration, and proteolysis resulting in extracellular matrix remodeling, immune escape, angiogenesis and lymphangiogenesis, and target ‘homing’. Most existing cancer treatments focus on killing tumor cells; however, such chemotherapeutic intervention leads to substantial toxicity to healthy cells and tissue. Since spread, or metastasis, of cancers is the primary cause of cancer-related mortalities, there is urgent need for agents that specifically inhibit or prevent signals that trigger metastasis.

Rho proteins are overexpressed in various tumors, including colon, breast, lung, testicular germ cell, and head and neck squamous-cell carcinoma (Sawyer, Expert Opin. Investig. Drugs., 13: 1-9, 2004; herein incorporated by reference in its entirety). The rho family of small GTP binding proteins plays important roles in many normal biological processes and in cancer (Schmidt and Hall, Genes Dev., 16:1587-1609, 2002; Burridge and Wennerberg, Cell, 116:167-179, 2004; each herein incorporated by reference in its entirety). This family includes three main groups: rho, rac, and cdc42. Rho is activated by numerous external stimuli including growth factor receptors, immune receptors, cell adhesion, and G protein coupled receptors (GPCRs) (Schmidt and Hall, Genes Dev., 16:1587-1609, 2002, Sah et al., Annu. Rev. Pharmacol. Toxicol., 40:459-489, 2000; each herein incorporated by reference in its entirety).

RhoA and rhoC play roles in metastasis (Clark et al., Nature 406:532-535, 2000; Ikoma et al., Clin Cancer Res 10:1192-1200, 2004; Shikada et al., Clin Cancer Res 9:5282-5286, 2003; Wu et al., Breast Cancer Res Treat 84:3-12, 2004; Hakem et al, Genes Dev 19:1974-9, 2005; each herein incorporated by reference in its entirety). Both rhoA and rac can regulate the function of the extracellular matrix (ECM) proteins, ezrin, moesin, and radixin, by the phosphorylation of ezrin via the rhoA pathway and the phosphorylation of the ezrin antagonist, neurofibromatosis 2, by the rac1 pathway (Shaw et al., Dev Cell 1:63-72, 2001; Matsui et al., J Cell Biol 140:647-657, 1998; each herein incorporated by reference in its entirety). These ECM proteins promote cell movement by utilizing the ECM receptor, CD44, to link the actin cytoskeleton with the plasma membrane. In addition, rhoA and rac1 regulate ECM remodeling by controlling the levels of matrix metalloproteinases (MMPs) or their antagonists, tissue inhibitors of metalloproteinases (TIMPs) (Bartolome et al., Cancer Res 64:2534-2543, 2004; herein incorporated by reference in its entirety). RhoA is also required for monocyte tail retraction during transendothelial migration, indicating a role in extravasation, which is a key process in metastasis (Worthylake et al., J Cell Biol 154:147-160, 2001; herein incorporated by reference in its entirety).

In addition to cytoskeletal effects, rhoA and rhoC induce gene transcription via the serum response factor, SRF. SRF is associated with cellular transformation and epithelial-mesenchymal transformation (Iwahara et al., Oncogene 22:5946-5957, 2003; Psichari et al., J Biol Chem 277:29490-29495, 2002; each herein incorporated by reference in its entirety). Rho activates SRF via release of the transcriptional coactivators mycardin-related transcription factors MRTF-A and MRTF-B (Cen et al., Mol Cell Biol 23:6597-6608, 2003; Miralles et al., Cell 113:329-342, 2003; Selvaraj and Prywes, J Biol Chem 278:41977-41987, 2003; each herein incorporated by reference in its entirety). MRTF-A is also known as megakaryoblastic leukemia 1 (MKL1, its gene name) and the gene name for MRTF-B is MKL2. MRTF-A, like the rhoGEF LARG, was first identified as a site of gene translocation in leukemia (megakaryoblastic leukemia) (Mercher et al., Genes Chromosomes Cancer 33:22-28, 2002; herein incorporated by reference in its entirety). The protein product of the translocated MKL1 gene is hyperactive compared to the wild-type protein. MRTF has also been called modified in acute leukemia (MAL) or BSAC (Miralles et al., Cell 113:329-342, 2003; Sasazuki et al., J Biol Chem 277:28853-28860, 2002; each herein incorporated by reference in its entirety). As a consequence of rho signaling, MRTF-A and -B translocate to the nucleus and binds SRF leading to the expression of c-fos which, along with c-jun, forms the transcription factor AP-1. The AP-1 transcription factor promotes the activity of various MMPs and other cell motility genes (Benbow and Brinckerhoff, Matrix Biol 15:519-526, 1997; herein incorporated by reference in its entirety). Expression of these genes leads to cancer cell invasion and metastasis. Also, MRTF-A was identified in an antiapoptosis screen for genes that abrogate tumor necrosis factor-induced cell death (Sasazuki et al., J Biol Chem 277:28853-28860, 2002; herein incorporated by reference in its entirety). MRTF proteins also control cancer cell migration and are essential for melanoma and breast cancer metastasis in mouse xenograft studies (Medjkane et al, Nat Cell Biol. 2009 March; 11(3):257-68 2009). MRTF-A and B together are overexpressed in 20% of breast cancer cell lines and mutated in ˜10% of pancreatic cancers and cutaneous melanomas (cbioportal.org; Gao et al. Sci. Signal. 2013 & Cerami et al. Cancer Discov. 2012). Thus, there is a strong link between rho-controlled gene transcription and cancer metastasis.

The relative contributions of rho and rac proteins in the metastatic phenotype has been studied (Sahai and Marshall, Nat Rev Cancer 2:133-142, 2002; Whitehead et al., Oncogene 20:1547-1555, 2001; each herein incorporated by reference in its entirety). Sahai and Marshall (Nat Cell Biol 5:711-719, 2003; herein incorporated by reference in its entirety) showed that different tumor cell lines exhibit different mechanisms of motility and invasion. In particular, 375 m2 melanoma and LS174T colon carcinoma cell lines showed striking “rounded” and “blebbed” morphology during invasion into Matrigel matrices. This invasion was entirely rho-dependent and was blocked by C3 exotoxin, the N17rho dominant negative protein, and a ROCK kinase inhibitor. In contrast, two other cell lines were blocked instead by a rac dominant negative mutation, but not rho or ROCK inhibitors. These latter two cell lines (BE colon carcinoma and SW962 squamous cell carcinoma) had elongated morphologies. A third line showed a mixed morphology and was blocked partially by both rho and rac inhibitors. Additionally, mice lacking rhoC have greatly reduced metastasis of virally-induced breast tumors to lung (Hakem et al, Genes Dev 19:1974-9, 2005; herein incorporated by reference in its entirety). Also, knock-down of SRF or its transcriptional coactivator MRTF reduced lung metastases from breast or melanoma xenografts (Medjkane et al, Nat Cell Biol. 11:257-68, 2009; herein incorporated in its entirety). Clearly there is important heterogeneity in mechanisms of tumor cell behavior that contributes to metastasis. It is widely recognized that cell growth and apoptosis mechanisms vary greatly among tumors, necessitating customized therapeutic approaches.

Nearly 40% of chronic diseases such as cirrhosis, heart failure, and diabetic nephropathy are characterized by fibrosis or excess deposition of extracellular matrix, including collagen. The poor clinical outcome of several orphan diseases (scleroderma or systemic sclerosis—SSc, idiopathic pulmonary fibrosis—IPF, primary sclerosing cholangitis-PSC, and IgA Nephropathy) is primarily determined by tissue fibrosis; there are absolutely no effective treatments despite their rapid and lethal clinical course.

Systemic sclerosis (scleroderma, SSc) is an orphan, multisystem autoimmune disorder that can cause fibrosis of the skin and internal organ systems (lungs, heart, kidneys, and gastrointestinal system). It has the highest case fatality rate of any rheumatic disease. SSc predominately affects women (Beyer et al., Arthritis Rheum 62: 2831-2844, 2010; Boukhalfa G, et al., Exp Nephrol 4: 241-247, 1996; Buhl A M, et al., J Biol Chem 270: 24631-24634, 1995; Chaqour et al., FEBS J 273: 3639-3649, 2006; Charles et al., Lancet 367: 1683-1691, 2006) and increases with age. The precise pathogenesis of SSc is yet to be defined but the major clinical features of SSc—collagen production, vascular damage and inflammation/autoimmunity—require environmental triggers and genetic effects which interact with the three cardinal features of the disease at several points (Charles et al., Lancet 367: 1683-1691, 2006). Generally, there is initial inflammation but fibrosis persists even after the inflammation has resolved or has been suppressed by medications (Beyer et al., Curr Opin Rheumatol 24: 274-280, 2012; Wynn T A, and Ramalingam T R. Nat Med 18: 1028-1040, 2012).

What is needed are new compositions and methods for targeted therapy to assist in the treatment and management of cancer and fibrosis.

### SUMMARY OF THE DISCLOSURE

The present disclosure relates to methods, compositions, and kits for the inhibition of signaling by members of the Rho GTPase family. Specifically, the disclosure relates to methods, compositions and kits for the inhibition of RhoA and/or RhoC transcriptional signaling and action of the transcription co-factors MRTF-A and/or MRTF-B. The disclosure finds use in treatment of Rho-mediated disease states (e.g., tumor metastasis and fibrosis), Rho-mediated biological conditions, and in cell signaling research.

For example, embodiments of the present disclosure provide a composition comprising a compound of structure I,

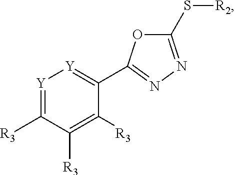

wherein Y is C—R3 or N, R2 is (CH2)nCOOR1, wherein each CH2 group may be optionally substituted, R1 is H or C1-C6 alkyl, n is an integer from 1 to 10, and R3 is the same or different and is H, a halide, an ether, or a straight or branched alkyl. In some embodiments, the composition comprises a compound of the structure:

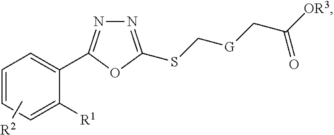

wherein R1 is halogen, or a C1-C5 straight or branched chain alkyl, C1-C3 alkyl-O; R2 is H, halogen, a C1-C5 straight or branched chain alkyl, or C1-C3 alkyl-O; R3 is H or C1-C3 alkyl; and G is (CH₂)ₙ wherein n=1 or 2. In some embodiments, when n=1 and R1 is Me, R2 is not 4-Me or H, and when n=1 and R1 is OMe, R2 is not H. In some embodiments, the composition is

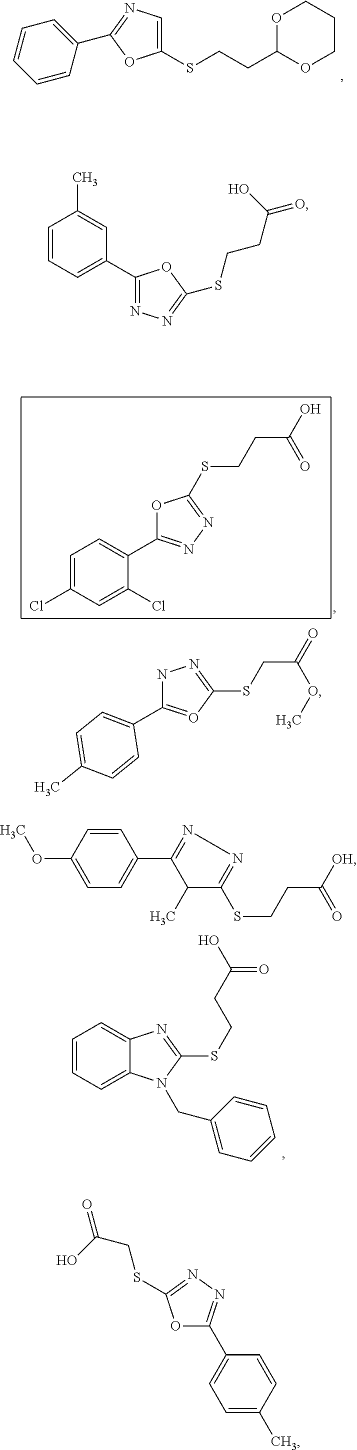

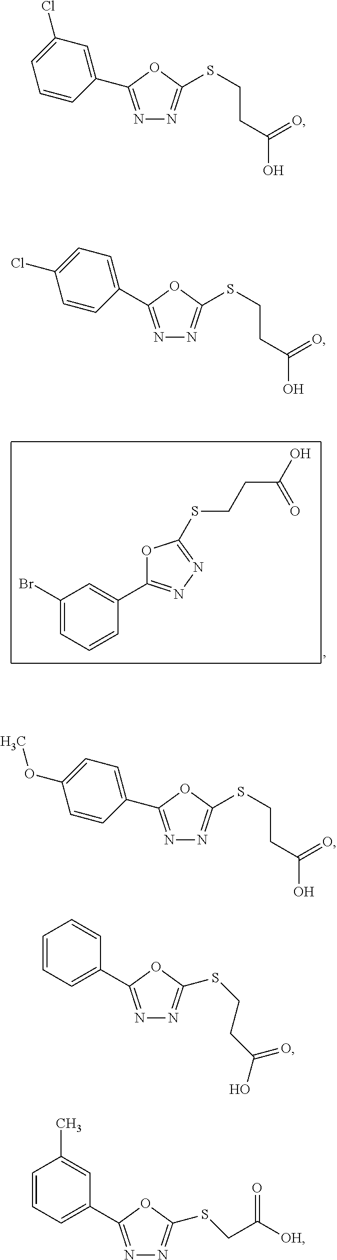 [SMILES: O=C(O)CCSc1nnc(-c2cccc(Cl)c2)o1 ; O=C(O)CCSc1nnc(-c2ccc(Cl)cc2)o1 ; O=C(O)CCSc1nnc(-c2cccc(Br)c2)o1 ; COc1ccc(-c2nnc(SCCC(=O)O)o2)cc1 ; O=C(O)CCSc1nnc(-c2ccccc2)o1 ; Cc1cccc(-c2nnc(SCC(=O)O)o2)c1]

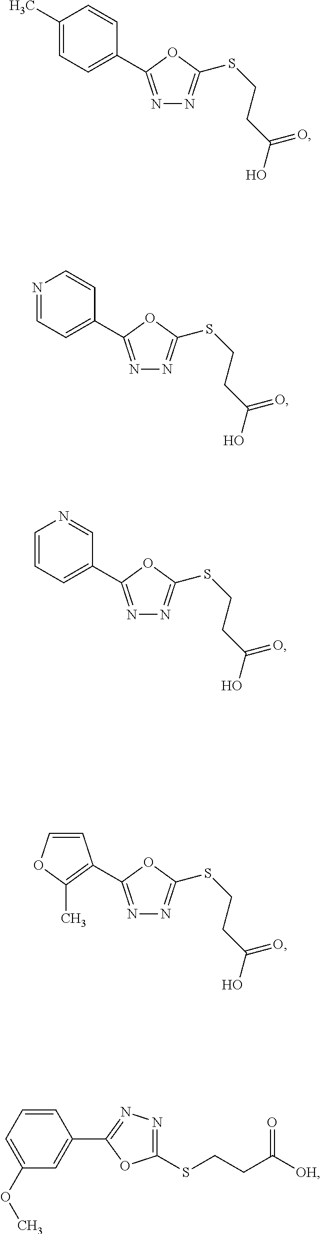 [SMILES: Cc1ccc(-c2nnc(SCCC(=O)O)o2)cc1 ; O=C(O)CCSc1nnc(-c2ccncc2)o1 ; O=C(O)CCSc1nnc(-c2cccnc2)o1 ; Cc1occc1-c1nnc(SCCC(=O)O)o1 ; COc1cccc(-c2nnc(SCCC(=O)O)o2)c1]

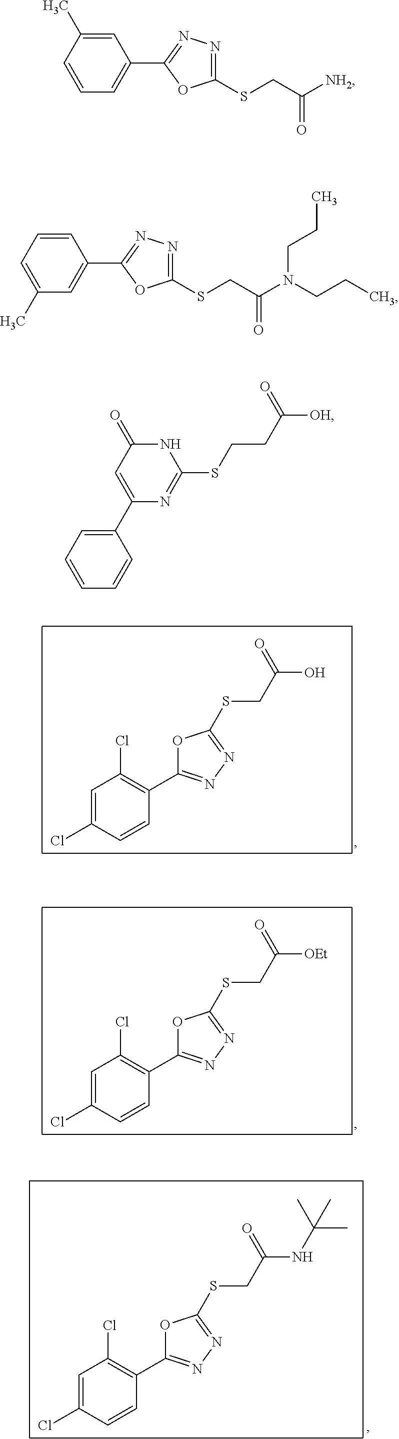 [SMILES: Cc1cccc(-c2nnc(SCC(N)=O)o2)c1 ; CCCN(CCC)C(=O)CSc1nnc(-c2cccc(C)c2)o1 ; O=C(O)CCSc1nc(-c2ccccc2)cc(=O)[nH]1 ; O=C(O)CSc1nnc(-c2ccc(Cl)cc2Cl)o1 ; CCOC(=O)CSc1nnc(-c2ccc(Cl)cc2Cl)o1 ; CC(C)(C)NC(=O)CSc1nnc(-c2ccc(Cl)cc2Cl)o1]

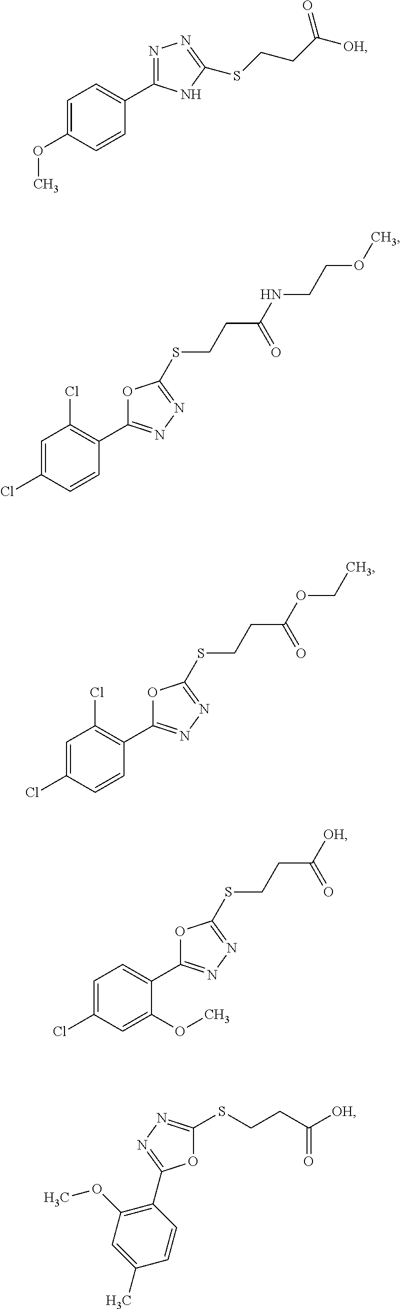 [SMILES: COc1ccc(-c2nnc(SCCC(=O)O)[nH]2)cc1 ; COCCNC(=O)CCSc1nnc(-c2ccc(Cl)cc2Cl)o1 ; CCOC(=O)CCSc1nnc(-c2ccc(Cl)cc2Cl)o1 ; COc1cc(Cl)ccc1-c1nnc(SCCC(=O)O)o1 ; COc1cc(C)ccc1-c1nnc(SCCC(=O)O)o1]

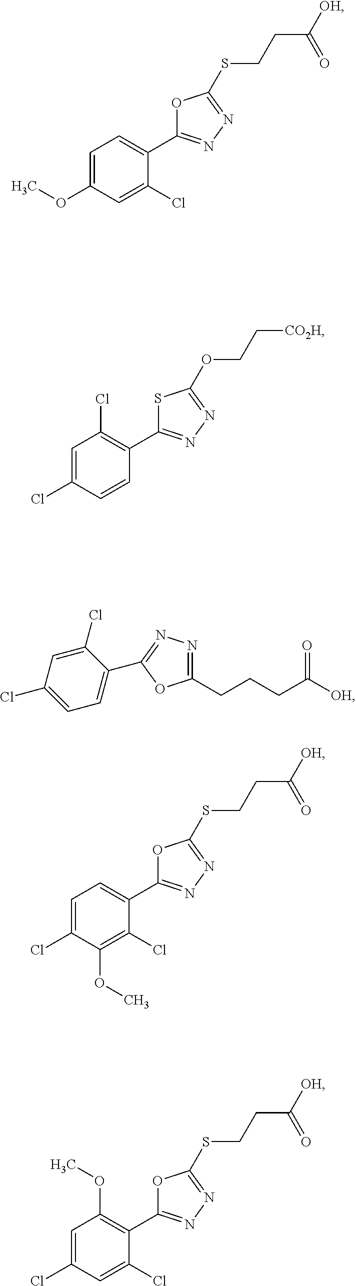 [SMILES: COc1ccc(-c2nnc(SCCC(=O)O)o2)c(Cl)c1 ; O=C(O)CCOc1nnc(-c2ccc(Cl)cc2Cl)s1 ; O=C(O)CCCc1nnc(-c2ccc(Cl)cc2Cl)o1 ; COc1c(Cl)ccc(-c2nnc(SCCC(=O)O)o2)c1Cl ; COc1cc(Cl)cc(Cl)c1-c1nnc(SCCC(=O)O)o1]

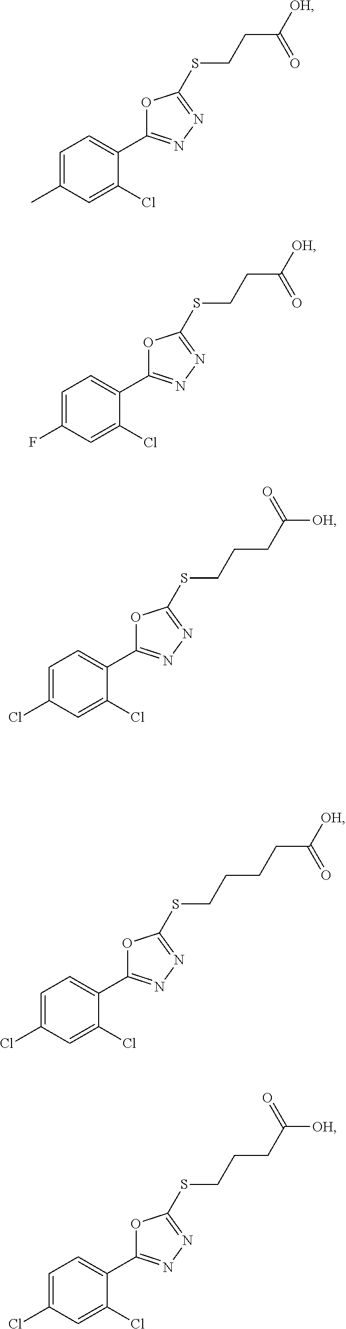 [SMILES: Cc1ccc(-c2nnc(SCCC(=O)O)o2)c(Cl)c1 ; O=C(O)CCSc1nnc(-c2ccc(F)cc2Cl)o1 ; O=C(O)CCCSc1nnc(-c2ccc(Cl)cc2Cl)o1 ; O=C(O)CCCCSc1nnc(-c2ccc(Cl)cc2Cl)o1 ; O=C(O)CCCSc1nnc(-c2ccc(Cl)cc2Cl)o1]

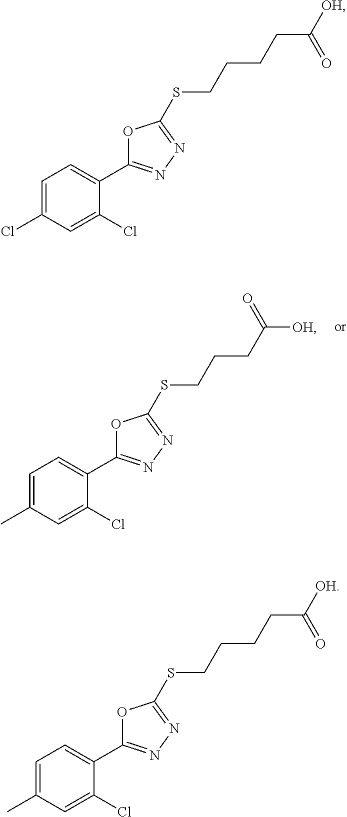 [SMILES: O=C(O)CCCCSc1nnc(-c2ccc(Cl)cc2Cl)o1 ; Cc1ccc(-c2nnc(SCCCC(=O)O)o2)c(Cl)c1 ; Cc1ccc(-c2nnc(SCCCCC(=O)O)o2)c(Cl)c1]

In some embodiments, the composition is in a pharmaceutically appropriate formulation for administration to a human subject. In some embodiments, the composition comprises a pharmaceutically acceptable carrier. In some embodiments, the composition comprises an additional agent (e.g., a chemotherapeutic agent, an anti-fibrotic agent, or an anti-inflammatory agent).

Embodiments of the present disclosure provide a method of treating or preventing a rho-mediated disease in a subject comprising administering any of the aforementioned compounds, or derivatives, stereoisomers, pharmaceutically acceptable salts, or mimetics thereof to the subject. Further embodiments provide the use of any of the aforementioned compounds, or derivatives, stereoisomers, pharmaceutically acceptable salts, or mimetics thereof in the treatment or prevention of a Rho mediated disease in a subject. In some embodiments, the rho-mediated disease is fibrosis (e.g., idiopathic pulmonary fibrosis), cancer, inflammation, inflammatory disease, Crohn's disease, pulmonary arterial hypertension, axon regeneration following nerve damage, Raynaud's phenomenon, cerebral vascular disease, cardiovascular disease, or erectile dysfunction. In some embodiments, the fibrosis is systemic sclerosis.

Additional embodiments of the present disclosure provide a method of treating or preventing a fibrotic disease (e.g., systemic sclerosis) in a subject comprising administering any of the aforementioned compounds, or derivatives, stereoisomers, pharmaceutically acceptable salts, or mimetics thereof to the subject.

Additional embodiments will be apparent to persons skilled in the relevant art based on the teachings contained herein.

### DESCRIPTION OF THE DRAWINGS

FIG. 1 shows a schematic model of multiple pro-fibrotic stimuli that all utilize the MRTF/SRF-regulated gene transcription.

FIG. 2 shows 3 Inhibitors of MRTF/SRF-regulated gene transcription. A) CCG-1423, B) Analog CCG-203971 (Series 1) with reduced acute cellular toxicity. C) CCG-58146 (Series 2) with markedly higher potency. D) ROCK inhibition does not fully inhibit G-protein-activated Luciferase (<50% max inhibition). E) inhibition by CCG-58150.

FIG. 3 shows highly potent inhibition of fibroblast signaling by CCG-58150 A) NIH3T3 cells were stimulated with LPA and CTGF mRNA analyzed by qRT-PCR. B-D) Human dermal fibroblasts were treated with TGFβ (10 ng/ml) for 3 days with various concentrations of CCG-58150.

FIG. 4 shows that CCG-203971 prevents fibrosis. A & B) CCG-203971 inhibits LPA-induced COL1A2 and ACTA2 (αSMA) mRNA in NIH-3T3 cells. C) Suppression of myofibroblast activation (αSMA staining) in TGFβ-induced normal and untreated SSc dermal fibroblasts. D) CCG-203971 prevents skin fibrosis.

FIG. 5 shows inhibition of cellular migration by 203971, 58150, and 58146.

FIG. 6 shows αSMA inhibition by 58150.

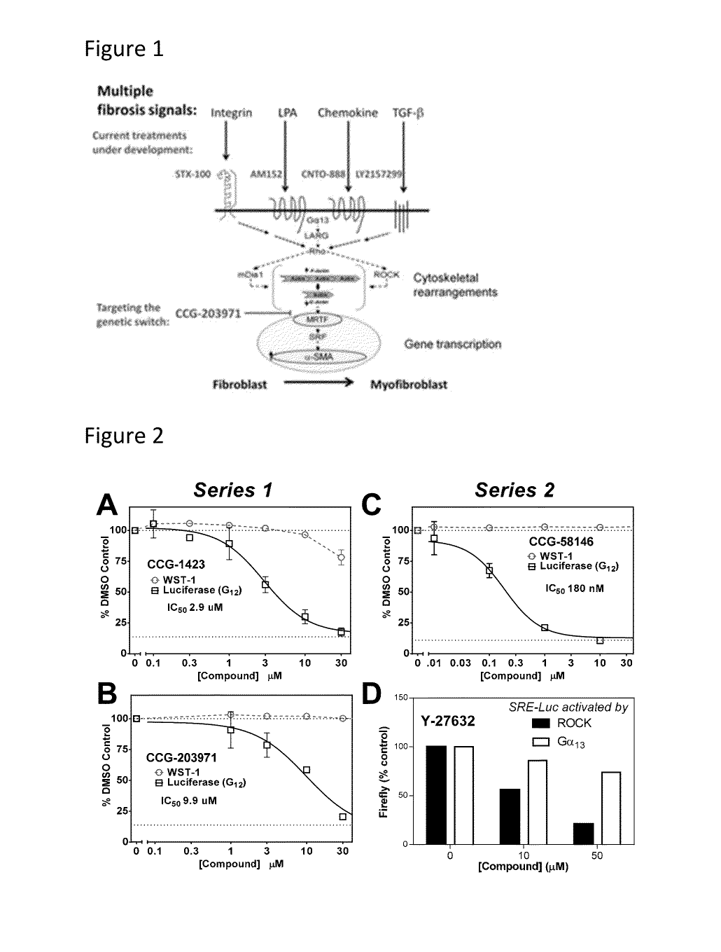

### DEFINITIONS

To facilitate an understanding of the present disclosure, a number of terms and phrases are defined below:

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “subject suspected of having fibrosis” refers to a subject that presents one or more symptoms indicative of a fibrotic disease. A subject suspected of having a fibrotic disease may also have one or more risk factors. A subject suspected of having fibrotic disease has generally not been tested for fibrotic disease. However, a “subject suspected of having fibrotic disease” encompasses an individual who has received a preliminary diagnosis but for whom a confirmatory test has not been done or for whom the level or severity of fibrotic disease is not known.

As used herein, the term “subject diagnosed with a fibrotic disease” refers to a subject who has been tested and found to have a fibrotic disease. As used herein, the term “initial diagnosis” refers to a test result of initial fibrotic disease that reveals the presence or absence of disease.

As used herein, the term “subject at risk for fibrotic disease” refers to a subject with one or more risk factors for developing a specific fibrotic disease. Risk factors include, but are not limited to, gender, age, genetic predisposition, environmental exposure, and previous incidents of fibrotic disease, preexisting non-fibrotic diseases, and lifestyle.

As used herein, the term “characterizing fibrotic disease in subject” refers to the identification of one or more properties of a fibrotic disease in a subject, including but not limited to, the presence of fibrotic disease, the type of fibrotic disease, or the severity of fibrotic disease.

As used herein, the term “providing a prognosis” refers to providing information regarding the impact of the presence of fibrotic disease on a subject's future health (e.g., expected morbidity or mortality, the likelihood of getting fibrotic disease, and the risk of the fibrotic disease progressing or spreading).

As used herein, the term “non-human animals” refers to all non-human animals including, but not limited to, vertebrates such as rodents, non-human primates, ovines, bovines, ruminants, lagomorphs, porcines, caprines, equines, canines, felines, aves, etc.

As used herein, the term “cell culture” refers to any in vitro culture of cells. Included within this term are continuous cell lines (e.g., with an immortal phenotype), primary cell cultures, transformed cell lines, finite cell lines (e.g., non-transformed cells), and any other cell population maintained in vitro.

As used herein, the term “eukaryote” refers to organisms distinguishable from “prokaryotes.” It is intended that the term encompass all organisms with cells that exhibit the usual characteristics of eukaryotes, such as the presence of a true nucleus bounded by a nuclear membrane, within which lie the chromosomes, the presence of membrane-bound organelles, and other characteristics commonly observed in eukaryotic organisms. Thus, the term includes, but is not limited to such organisms as fungi, protozoa, and animals (e.g., humans).

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

The terms “test compound” and “candidate compound” refer to any chemical entity, pharmaceutical, drug, and the like that is a candidate for use to treat or prevent a disease, illness, sickness, or disorder of bodily function (e.g., fibrosis or cancer). Test compounds comprise both known and potential therapeutic compounds. A test compound can be determined to be therapeutic by screening using the screening methods of the present disclosure.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, and industrial samples. Such examples are not however to be construed as limiting the sample types applicable to the present disclosure.

As used herein, the terms “rho” or “rho proteins” refer to the narrowly defined rho subfamily that includes rhoA, rhoB, rhoC, etc. and is described in (Sahai and Marshall, Nat Rev Cancer 2:133-142, 2002; herein incorporated by reference in its entirety). These terms do not refer to the larger rho family (i.e. do not refer to rac and cdc42). The term “Rho family” is used to designate the larger group including the three rho subfamilies (rho, rac, and cdc42).

As used herein, the term “effective amount” refers to the amount of a compound (e.g., a rho-inhibiting compound having a structure presented above or elsewhere described herein) sufficient to effect beneficial or desired results. An effective amount can be administered in one or more administrations, applications or dosages and is not limited to or intended to be limited to a particular formulation or administration route.

As used herein, the term “co-administration” refers to the administration of at least two agent(s) (e.g., a rho-inhibiting compound having a structure presented above or elsewhere described herein) or therapies to a subject. In some embodiments, the co-administration of two or more agents/therapies is concurrent. In other embodiments, a first agent/therapy is administered prior to a second agent/therapy. Those of skill in the art understand that the formulations and/or routes of administration of the various agents/therapies used may vary. The appropriate dosage for co-administration can be readily determined by one skilled in the art. In some embodiments, when agents/therapies are co-administered, the respective agents/therapies are administered at lower dosages than appropriate for their administration alone. Thus, co-administration is especially desirable in embodiments where the co-administration of the agents/therapies lowers the requisite dosage of a known potentially harmful (e.g., toxic) agent(s).

As used herein, the term “pharmaceutical composition” refers to the combination of an active agent with a carrier, inert or active, making the composition especially suitable for diagnostic or therapeutic use in vivo, in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants. (See e.g., Martin, Remington's Pharmaceutical Sciences, 15th Ed., Mack Publ. Co., Easton, Pa. [1975]).

As used herein, the term “pharmaceutically acceptable salt” refers to any pharmaceutically acceptable salt (e.g., acid or base) of a compound of the present disclosure which, upon administration to a subject, is capable of providing a compound of this disclosure or an active metabolite or residue thereof. As is known to those of skill in the art, “salts” of the compounds of the present disclosure may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the disclosure and their pharmaceutically acceptable acid addition salts.

Examples of bases include, but are not limited to, alkali metals (e.g., sodium) hydroxides, alkaline earth metals (e.g., magnesium), hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present disclosure compounded with a suitable cation such as Na⁺, NH₄⁺, and NW₄⁺ (wherein W is a C₁₋₄ alkyl group), and the like.

For therapeutic use, salts of the compounds of the present disclosure are contemplated as being pharmaceutically acceptable. However, salts of acids and bases that are non-pharmaceutically acceptable may also find use, for example, in the preparation or purification of a pharmaceutically acceptable compound.

As used herein, the term “instructions for administering said compound to a subject,” and grammatical equivalents thereof, includes instructions for using the compositions contained in a kit for the treatment of conditions characterized by viral infection (e.g., providing dosing, route of administration, decision trees for treating physicians for correlating patient-specific characteristics with therapeutic courses of action). The rho-inhibiting compounds of the present disclosure (e.g. as shown in structures above and elsewhere presented herein) can be packaged into a kit, which may include instructions for administering the compounds to a subject.

As used herein, the term “chemical moiety” refers to any chemical compound containing at least one carbon atom. Examples of chemical moieties include, but are not limited to, aromatic chemical moieties, chemical moieties comprising sulfur, chemical moieties comprising nitrogen, hydrophilic chemical moieties, and hydrophobic chemical moieties.

As used herein, the term “heteroaryl” refers to an aromatic ring with at least one carbon replaced by O, S or N.

As used herein, the term “aliphatic” represents the groups including, but not limited to, alkyl, alkenyl, alkynyl, alicyclic.

As used herein, the term “aryl” represents a single aromatic ring such as a phenyl ring, or two or more aromatic rings (e.g., bisphenyl, naphthalene, anthracene), or an aromatic ring and one or more non-aromatic rings. The aryl group can be optionally substituted with a lower aliphatic group (e.g., alkyl, alkenyl, alkynyl, or alicyclic). Additionally, the aliphatic and aryl groups can be further substituted by one or more functional groups including, but not limited to, —NH₂, —NHCOCH₃, —OH, lower alkoxy (C₁-C₄), halo (—F, —Cl, —Br, or —I).

As used herein, the term “substituted aliphatic,” refers to an alkane, alkene, alkyne, or alicyclic moiety where at least one of the aliphatic hydrogen atoms has been replaced by, for example, a halogen, an amino, a hydroxy, a nitro, a thio, a ketone, an aldehyde, an ester, an amide, a lower aliphatic, a substituted lower aliphatic, or a ring (aryl, substituted aryl, cycloaliphatic, or substituted cycloaliphatic, etc.). Examples of such include, but are not limited to, 1-chloroethyl and the like.

As used herein, the term “substituted aryl” refers to an aromatic ring or fused aromatic ring system consisting of at least one aromatic ring, and where at least one of the hydrogen atoms on a ring carbon has been replaced by, for example, a halogen, an amino, a hydroxy, a nitro, a thio, a ketone, an aldehyde, an ester, an amide, a lower aliphatic, a substituted lower aliphatic, or a ring (aryl, substituted aryl, cycloaliphatic, or substituted cycloaliphatic). Examples of such include, but are not limited to, hydroxyphenyl and the like.

As used herein, the term “cycloaliphatic” refers to an aliphatic structure containing a fused ring system. Examples of such include, but are not limited to, decalin and the like.

As used herein, the term “substituted cycloaliphatic” refers to a cycloaliphatic structure where at least one of the aliphatic hydrogen atoms has been replaced by a halogen, a nitro, a thio, an amino, a hydroxy, a ketone, an aldehyde, an ester, an amide, a lower aliphatic, a substituted lower aliphatic, or a ring (aryl, substituted aryl, cycloaliphatic, or substituted cycloaliphatic). Examples of such include, but are not limited to, 1-chlorodecalyl, bicyclo-heptanes, octanes, and nonanes (e.g., nonrbornyl) and the like.

As used herein, the term “heterocyclic” represents, for example, an aromatic or nonaromatic ring containing one or more heteroatoms. The heteroatoms can be the same or different from each other. Examples of heteratoms include, but are not limited to nitrogen, oxygen and sulfur. Aromatic and nonaromatic heterocyclic rings are well-known in the art. Some nonlimiting examples of aromatic heterocyclic rings include pyridine, pyrimidine, indole, purine, quinoline and isoquinoline. Nonlimiting examples of nonaromatic heterocyclic compounds include piperidine, piperazine, morpholine, pyrrolidine and pyrazolidine. Examples of oxygen containing heterocyclic rings include, but not limited to furan, oxirane, 2H-pyran, 4H-pyran, 2H-chromene, and benzofuran. Examples of sulfur-containing heterocyclic rings include, but are not limited to, thiophene, benzothiophene, and parathiazine. Examples of nitrogen containing rings include, but not limited to, pyrrole, pyrrolidine, pyrazole, pyrazolidine, imidazole, imidazoline, imidazolidine, pyridine, piperidine, pyrazine, piperazine, pyrimidine, indole, purine, benzimidazole, quinoline, isoquinoline, triazole, and triazine. Examples of heterocyclic rings containing two different heteroatoms include, but are not limited to, phenothiazine, morpholine, parathiazine, oxazine, oxazole, thiazine, and thiazole. The heterocyclic ring is optionally further substituted with one or more groups selected from aliphatic, nitro, acetyl (i.e., —C(═O)—CH₃), or aryl groups.

As used herein, the term “substituted heterocyclic” refers to a heterocylic structure where at least one of the ring carbon atoms is replaced by oxygen, nitrogen, phosphorous, or sulfur, and where at least one of the aliphatic hydrogen atoms has been replaced by a halogen, hydroxy, a thio, nitro, an amino, a ketone, an aldehyde, an ester, an amide, a lower aliphatic, a substituted lower aliphatic, or a ring (aryl, substituted aryl, cycloaliphatic, or substituted cycloaliphatic). Examples of such include, but are not limited to 2-chloropyranyl.

As used herein, the term “a chemical moiety that participates in hydrogen bonding” represents a group that can accept or donate a proton to form a hydrogen bond thereby. Some specific non-limiting examples of moieties that participate in hydrogen bonding include fluoro-containing groups, oxygen-containing groups, sulfur-containing groups, and nitrogen-containing groups that are well-known in the art (e.g., a hydroxyl group, a phenol group, an amide group, a sulfonamide group, an amine group, an aniline group, a benzimidizalone group, a carbamate group, and an imidizole group). Some examples of oxygen-containing groups that participate in hydrogen bonding include: hydroxy, lower alkoxy, lower carbonyl, lower carboxyl, lower ethers and phenolic groups. The qualifier “lower” as used herein refers to lower aliphatic groups (C₁-C₄) to which the respective oxygen-containing functional group is attached. Thus, for example, the term “lower carbonyl” refers to inter alia, formaldehyde, acetaldehyde. Some nonlimiting examples of nitrogen-containing groups that participate in hydrogen bond formation include amino and amido groups. Additionally, groups containing both an oxygen and a nitrogen atom can also participate in hydrogen bond formation. Examples of such groups include nitro, N-hydroxy and nitrous groups. It is also possible that the hydrogen-bond acceptor in the present disclosure can be the in electrons of an aromatic ring.

As used herein, the term “derivative” of a compound refers to a chemically modified compound wherein the chemical modification takes place at a functional group of the compound (e.g., aromatic ring). Such derivatives include, but are not limited to, esters of alcohol-containing compounds, esters of carboxy-containing compounds, amides of amine-containing compounds, amides of carboxy-containing compounds, imines of amino-containing compounds, acetals of aldehyde-containing compounds, ketals of carbonyl-containing compounds, and the like.

As used herein, the term “toxic” refers to any detrimental or harmful effects on a cell or tissue as compared to the same cell or tissue prior to the administration of the toxicant.

### DETAILED DESCRIPTION OF THE DISCLOSURE

The present disclosure relates to methods, compositions, and kits for the inhibition of signaling by members of the Rho GTPase family. Specifically, the disclosure relates to methods, compositions and kits for the inhibition of RhoA and/or RhoC transcriptional signaling and action of the transcription co-factors MRTF-A and/or MRTF-B. The disclosure finds use in treatment of Rho-mediated disease states (e.g., tumor metastasis and fibrosis), Rho-mediated biological conditions, and in cell signaling research.

A central feature of virtually all diseases of fibrosis is the activation of fibroblasts and differentiation into myofibroblasts (Boukhalfa et al., 1996. Exp Nephrol 4:241-247; Sappino et al., 1990. Am J Pathol 137:585-591; Zhang et al., 1996. Am J Pathol 148:527-537; Gilbane et al., 2013. Arthritis Res Ther 15:215; Hinz et al., 2012. Am J Pathol 180:1340-1355; Hu et al., 2013. Curr Opin Rheumatol 25:71-77; Wynn et al., 2012. Nat Med 18:1028-1040; Beyer et al., 2010. Arthritis Rheum 62:2831-2844). These cells proliferate rapidly and produce abundant extracellular matrix (Hu et al., supra; Small, E. M. 2012. J Cardiovasc Transl Res 5:794-804; Tomasek et al., 2008. Faseb Journal 22; Zhou et al., 2013. J Clin Invest 123:1096-1108). The expression of alpha-smooth muscle actin (a-SMA) is a widely recognized marker for myofibroblasts but it also contributes to their maintenance (Boukhalfa et al., supra; Sappino et al., supra; Zhang et al., supra; Gilbane et al., supra; Hu et al., supra; Tomasek, et al., 2002. Nat Rev Mol Cell Biol 3:349-363). There are multiple signaling pathways that induce myofibroblast transition. TGFb is a critical mediator but lysophosphatidic acid (LPA), chemokines, endothelin, thombin, angiotensin, and connective tissue growth factor (CTGF) have all been implicated (Gilbane et al., 2013. Arthritis Res Ther 15:215; Wynn et al. supra: Beyer et al., 2012. Curr Opin Rheumatol 24:274-280). Additionally, tissue stiffness has been identified as a positive feedback mechanism that leads to further myofibroblast activation—probably through integrin and focal adhesion kinase (FAK) mechanisms (Wynn et al., supra; (Boukhalfa et al., supra; Sappino et al., supra; Zhang et al., supra; Gilbane et al., supra; Hu et al., supra; Tomasek, et al., 2002. Nat Rev Mol Cell Biol 3:349-363; Tomasek et al., 2008. Faseb Journal 22.). Development of anti-fibrotic therapies targeting the contractile and collagen synthetic functions of mesenchymal cells has been limited by the presence of these distinct pro-fibrotic pathways. So there is a need to identify compounds that target not one specific mediator or receptor but a common mechanism that is utilized by multiple pro-fibrotic stimuli in modulating mesenchymal cell functions.

Emerging evidence implicates gene transcription induced by serum response factor (SRF) as a critical driver of myofibroblast activation by nearly all of these mechanisms (Small. E. M. 2012. J Cardiovasc Transl Res 5:794-804; Sandbo et al., 2011. Am J Physiol Lung Cell Mol Physiol 301:L656-666; Small et al., 2010. Circ Res 107:294-304). Indeed, key genes involved in fibrosis are direct SRF targets including CTGF, COLI A2, and even ACTA2, the gene for a-SMA itself (Sakai et al., 2013. FASEB J.; Yang et al., 2003. Am J Respir Cell Mol Biol 29:583-590). This concept of a central role for SRF helps rationalize the complex signaling mechanisms that have been implicated in fibrosis. SRF-regulated gene expression is dependent on Rho-GTPase stimulated nuclear localization of its transcriptional coactivator MRTF. RhoA appears to be a convergent downstream mediator activated by virtually all of the signal pathways controlling the myofibroblast transition (Small et al., 2012 supra; Tomasek et al., 2008, supra; Small et all, 2010, supra; Sandbo et al., 2009, supra). G protein-coupled receptors for LPA. endothelin, thrombin, angiotensin and even chemokines activate RhoA (Seasholtz et al., 1999. Mol Pharmacol 55:949-956; Kranenburg et al., 1999. Mol Biol Cell 10:1851-1857). Other factors important in fibrosis including TGFβ and FAK also modulate MRTF/SRF activity through activation of Rho signaling and actin dynamics (Sakai et al, 2013, supra; Crider et al., 2011. J Invest Dermatol 131:2378-2385; Huang et al., 2012. Am J Respir Cell Mol Biol 47:340-348) which in turn drives expression of connective tissue growth factor (CTGF or CCN2) which synergizes with TGFβ in its pro-fibrotic actions (23-25). Thus, MRTF/SRF pathway serves as a common mediator of divergent fibrotic pathways and offers a therapeutic target in drug development for fibrosis

Recent studies show that pharmacological targeting of Rho/MRTF-regulated gene transcription can prevent or reverse fibrosis in both chemical and genetic models of skin (PMID: 24706986) and lung (PMID: 25681733) fibrosis.

Accordingly, embodiments of the present disclosure provide compositions and methods for targeting rho as a treatment for fibrosis, cancer, and inflammatory disease.

### I. Rho Cell Signaling Pathway

The mechanism of signaling by heterotrimeric G protein-coupled receptors that activate rho has been described (Sah et al., Annu Rev Pharmacol Toxicol 40:459-489, 2000; herein incorporated by reference in its entirety). The discovery of a family of unique rho guanine nucleotide exchange factors (rhoGEFs), p115rhoGEF (Hart et al., J Biol Chem 271:25452-25458, 1996; herein incorporated by reference in its entirety), PDZrhoGEF (Fukuhara et al., J Biol Chem 274:5868-5879, 1999; herein incorporated by reference in its entirety), and LARG (Leukemia-associated rhoGEF) (Kourlas et al., Proc Natl Acad Sci USA 97:2145-2150, 2000; herein incorporated by reference in its entirety) suggested a common mechanism. They contain a regulator of G protein signaling (RGS) domain that binds activated Gα12 (Suzuki et al., Proc Natl Acad Sci USA 100:733-738, 2003; herein incorporated by reference in its entirety) and Gα13 (Hart et al., Science 280:2112-2114, 1998; herein incorporated by reference in its entirety) causing rhoGEF activation. Thus, the RGS-rhoGEFs appear to serve as effectors of activated Gα12/13 and as molecular bridges between the heterotrimeric G protein alpha subunits and rho. This is a novel action of an RGS-domain containing protein, since they typically inhibit GPCR responses (Neubig and Siderovski, Nat Rev Drug Discov 1:187-197, 2002; herein incorporated by reference in its entirety). A role for RGS-rhoGEF proteins in cellular rho signaling by GPCRs, such as those for thrombin and lysophosphatidic acid (LPA), has been shown by studies with dominant negative constructs (Mao et al., Proc Natl Acad Sci USA 95:12973-12976, 1998; Majumdar et al., J Biol Chem 274:26815-26821, 1999; each herein incorporated by reference in its entirety) and inhibition of signaling by expression of the RGS-domains which act as Gα12/13 inhibitors (Fukuhara et al., FEBS Lett 485:183-188, 2000; herein incorporated by reference in its entirety).

Direct evidence that these RGS-RhoGEF proteins mediate GPCR signals and information about which rhoGEF(s) are downstream of which receptors has been shown (Wang et al., J Biol Chem., 279(28):28831-28834, 2004; herein incorporated by reference in its entirety). Experimentally, rho activation is detected directly by measurements of GTP-bound active rho precipitated from cell lysates with effector fusion proteins such as GST-rhotekin (Reid et al., J Biol Chem 271:13556-13560, 1996; herein incorporated by reference in its entirety) or indirectly by any number of functional readouts. The 1321N1 astrocytoma cell system is a well-studied model of thrombin-induced rho activation (Majumdar et al., J Biol Chem 273:10099-10106, 1998; herein incorporated by reference in its entirety). Thrombin induces both cell rounding and enhanced cell proliferation in these astrocytoma cells by mechanisms that are independent of known second messengers but are blocked by rho inhibitors.

Wang et al. (J Biol Chem., 279(28):28831-28834, 2004; herein incorporated by reference in its entirety) used HEK293T cells and an aggressive, metastatic, human prostate cancer cell line, PC-3, to test the role of the three RGS-rhoGEFs (LARG, p115-, and PDZrhoGEF) in receptor signaling. HEK293 and PC-3 cells express all three of these proteins. Transcriptional expression of PDZrhoGEF and LARG exceeds that of p115. PC-3 cells over-express the thrombin receptor (PAR1) and have an increased propensity to metastasize to bone compared to lines that have lower PAR1 expression (Cooper et al., Cancer 97:739-747, 2003; herein incorporated by reference in its entirety). To demonstrate a role for rhoGEFs in GPCR signaling and to define the specificity of their actions, it was shown by siRNA targeting that LARG mediates thrombin responses while the LPA response is mediated by PDZrhoGEF. This was the first direct demonstration of a role for an RGS-rhoGEF in G protein coupled receptor signaling. Furthermore, it pinpointed critical RGS-rhoGEFs (LARG and PDZrhoGEF) and allowed use of rhoGEFs in screening for modulators of rho-stimulated activities.

Development of synthetic RNAi molecules against the three members of this protein family showed that in PC-3 cells, the thrombin receptor (PAR1) utilized LARG while the LPA receptor utilized PDZ-rhoGEF for inducing cell rounding (Wang et al., J Biol Chem., 279(28):28831-28834, 2004; herein incorporated by reference in its entirety). In addition, direct measurements of thrombin-induced rho activation in HEK293T cells by GST-rhotekin pulldown also demonstrated a dependence on LARG. In the course of these studies, the rho transcription reporter method that uses the rho-specific SRE-Luciferase was developed. This transcriptional reporter method, described in detail therein, was used for screening a small chemical library for possible rho inhibitors (Evelyn et al., Mol Canc. Ther. 6:2249-60, 2007; herein incorporated by reference in its entirety). Additional inhibitors were identified as described below.

### II. Compositions

Embodiments of the present disclosure provide compositions that target and inhibit rho (e.g., for use in therapeutic, research, and screening applications). Exemplary compounds are described herein.

In some embodiments, the compound is a compound of structure I,

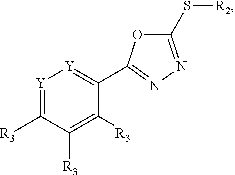

wherein Y is C—R3 or N, R2 is (CH2)nCOOR1, wherein each CH2 group may be optionally substituted, R1 is H or C1-C6 alkyl, n is an integer from 1 to 10, and R3 is the same or different and is H, a halide, an ether, or a straight or branched alkyl. In some embodiments, the composition comprises a compound of the structure:

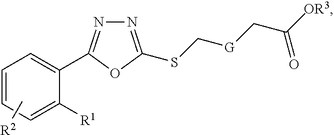

wherein R1 is halogen, or a C1-C5 straight or branched chain alkyl, C1-C3 alkyl-O; R2 is H, halogen, a C1-C5 straight or branched chain alkyl, or C1-C3 alkyl-O; R3 is H or C1-C3 alkyl; and G is (CH₂)ₙ wherein n=1 or 2. In some embodiments, when n=1 and R1 is Me, R2 is not 4-Me or H, and when n=1 and R1 is OMe, R2 is not H.

In some embodiments, the composition is, for example:

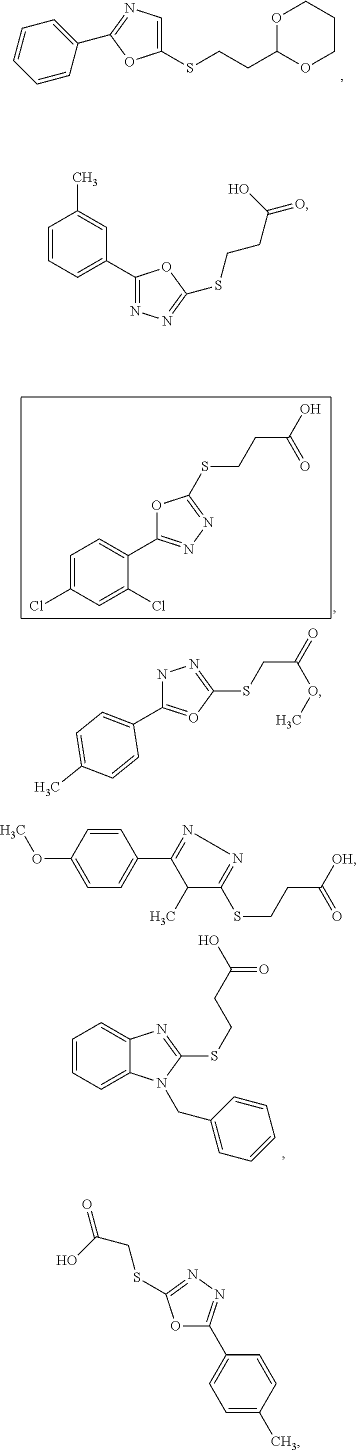

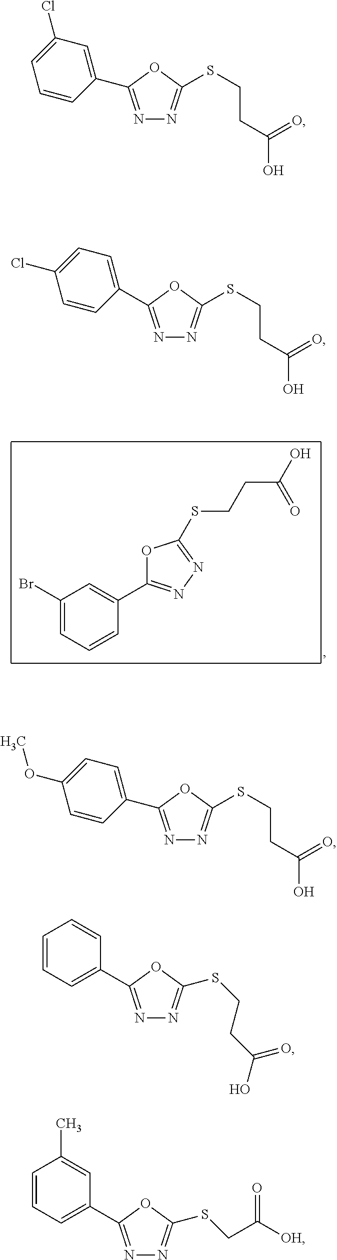 [SMILES: O=C(O)CCSc1nnc(-c2cccc(Cl)c2)o1 ; O=C(O)CCSc1nnc(-c2ccc(Cl)cc2)o1 ; O=C(O)CCSc1nnc(-c2cccc(Br)c2)o1 ; COc1ccc(-c2nnc(SCCC(=O)O)o2)cc1 ; O=C(O)CCSc1nnc(-c2ccccc2)o1 ; Cc1cccc(-c2nnc(SCC(=O)O)o2)c1]

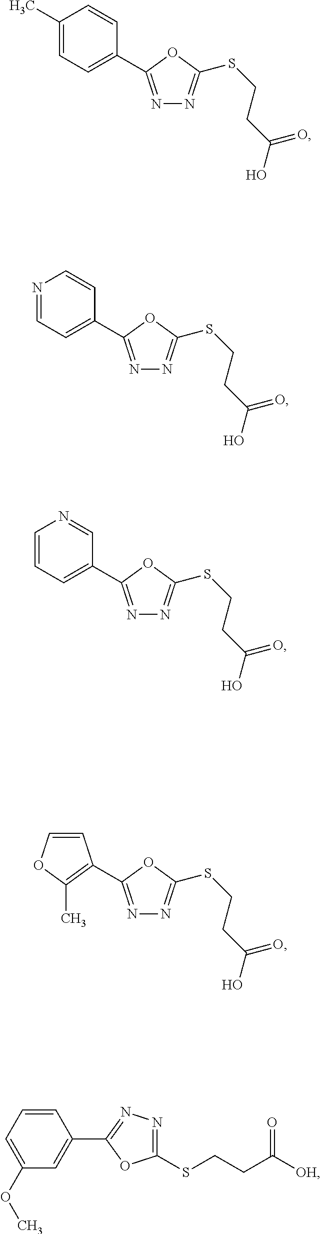 [SMILES: Cc1ccc(-c2nnc(SCCC(=O)O)o2)cc1 ; O=C(O)CCSc1nnc(-c2ccncc2)o1 ; O=C(O)CCSc1nnc(-c2cccnc2)o1 ; Cc1occc1-c1nnc(SCCC(=O)O)o1 ; COc1cccc(-c2nnc(SCCC(=O)O)o2)c1]

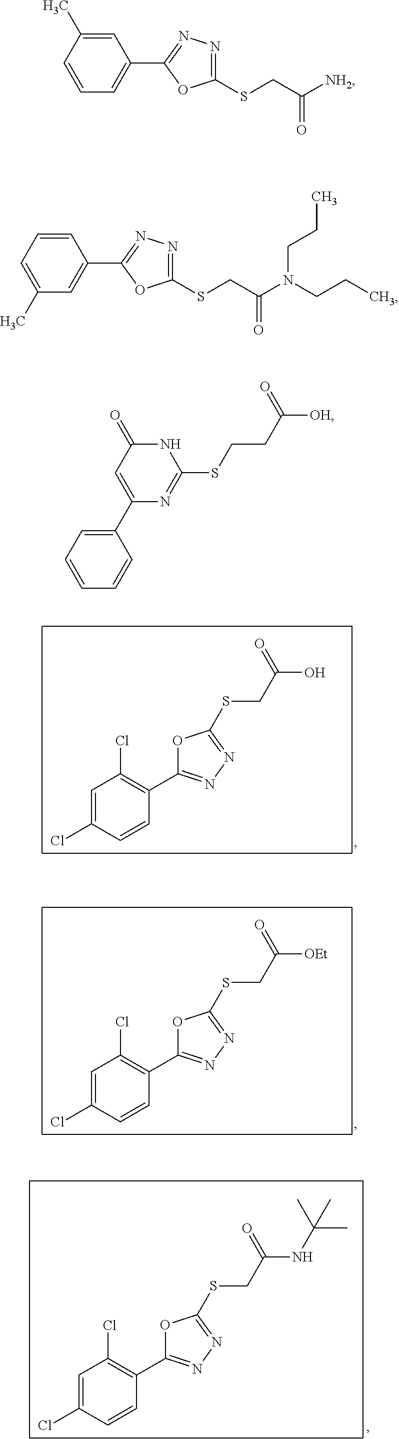 [SMILES: Cc1cccc(-c2nnc(SCC(N)=O)o2)c1 ; CCCN(CCC)C(=O)CSc1nnc(-c2cccc(C)c2)o1 ; O=C(O)CCSc1nc(-c2ccccc2)cc(=O)[nH]1 ; O=C(O)CSc1nnc(-c2ccc(Cl)cc2Cl)o1 ; CCOC(=O)CSc1nnc(-c2ccc(Cl)cc2Cl)o1 ; CC(C)(C)NC(=O)CSc1nnc(-c2ccc(Cl)cc2Cl)o1]

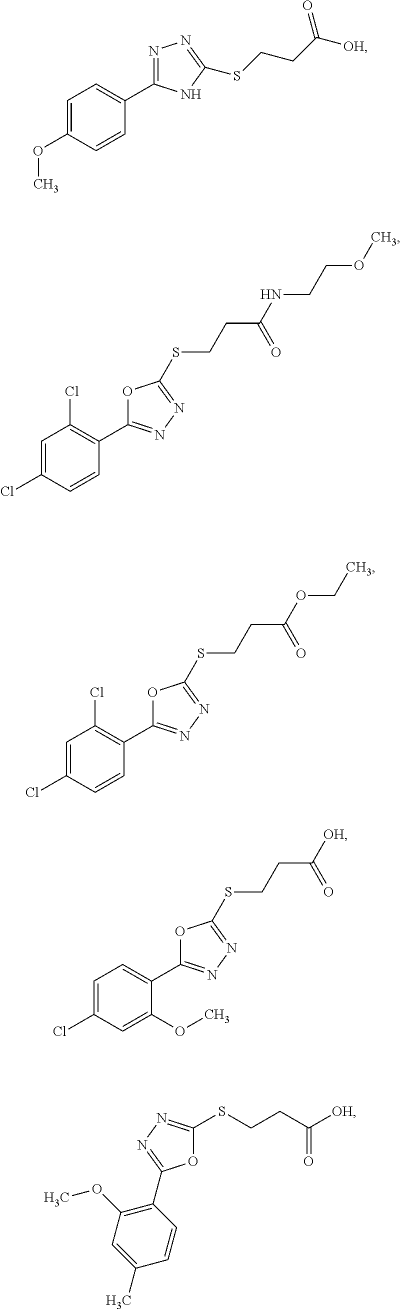 [SMILES: COc1ccc(-c2nnc(SCCC(=O)O)[nH]2)cc1 ; COCCNC(=O)CCSc1nnc(-c2ccc(Cl)cc2Cl)o1 ; CCOC(=O)CCSc1nnc(-c2ccc(Cl)cc2Cl)o1 ; COc1cc(Cl)ccc1-c1nnc(SCCC(=O)O)o1 ; COc1cc(C)ccc1-c1nnc(SCCC(=O)O)o1]

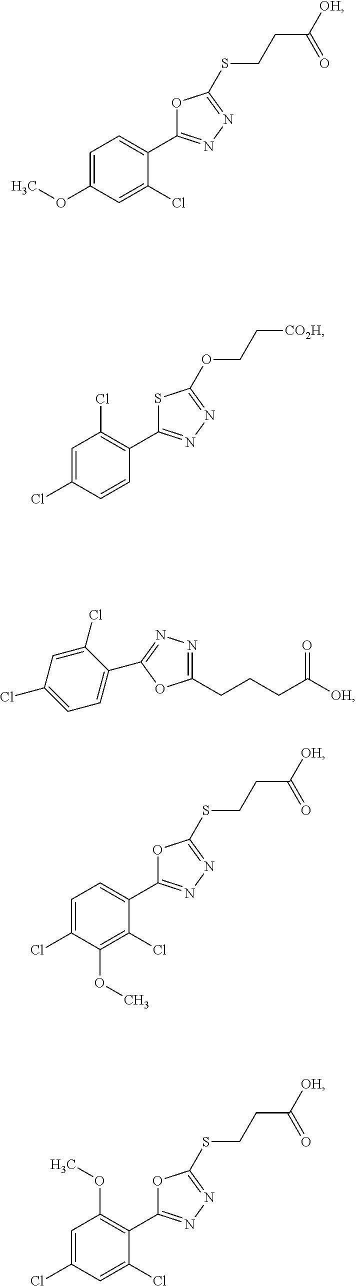 [SMILES: COc1ccc(-c2nnc(SCCC(=O)O)o2)c(Cl)c1 ; O=C(O)CCOc1nnc(-c2ccc(Cl)cc2Cl)s1 ; O=C(O)CCCc1nnc(-c2ccc(Cl)cc2Cl)o1 ; COc1c(Cl)ccc(-c2nnc(SCCC(=O)O)o2)c1Cl ; COc1cc(Cl)cc(Cl)c1-c1nnc(SCCC(=O)O)o1]

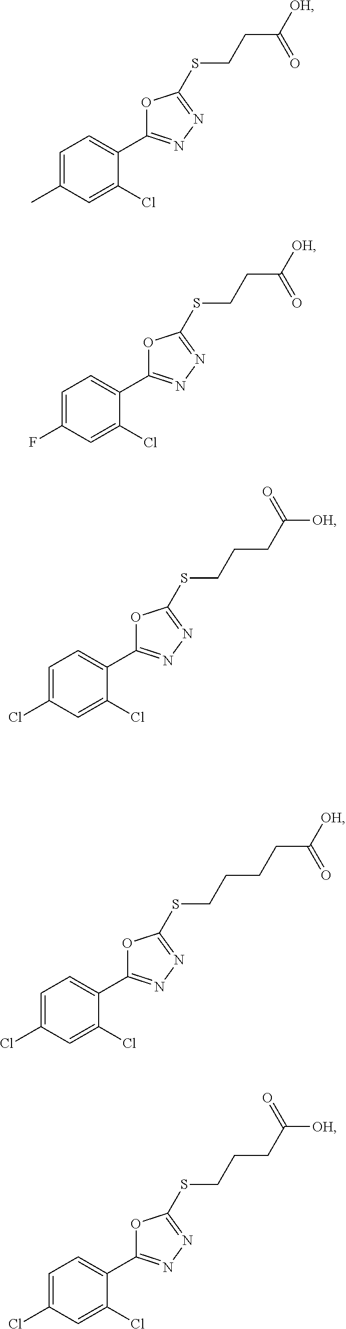 [SMILES: Cc1ccc(-c2nnc(SCCC(=O)O)o2)c(Cl)c1 ; O=C(O)CCSc1nnc(-c2ccc(F)cc2Cl)o1 ; O=C(O)CCCSc1nnc(-c2ccc(Cl)cc2Cl)o1 ; O=C(O)CCCCSc1nnc(-c2ccc(Cl)cc2Cl)o1 ; O=C(O)CCCSc1nnc(-c2ccc(Cl)cc2Cl)o1]

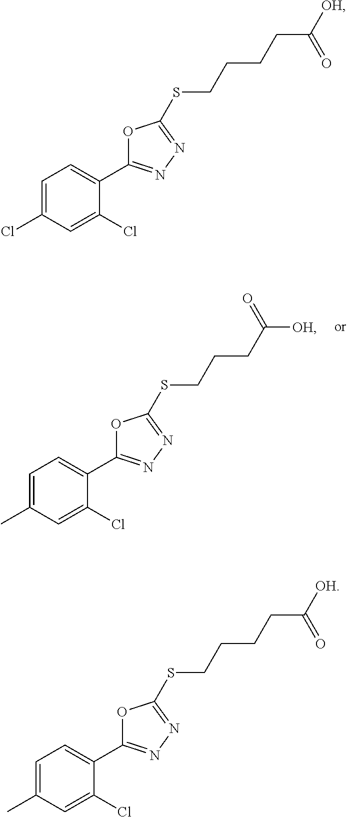 [SMILES: O=C(O)CCCCSc1nnc(-c2ccc(Cl)cc2Cl)o1 ; Cc1ccc(-c2nnc(SCCCC(=O)O)o2)c(Cl)c1 ; Cc1ccc(-c2nnc(SCCCCC(=O)O)o2)c(Cl)c1]

The present disclosure further contemplates derivatives, stereoisomers, mimetics, and salts of the compounds described herein.

Where clinical applications are contemplated, in some embodiments of the present disclosure, the rho-inhibiting compositions of the present disclosure are prepared as part of a pharmaceutical composition in a form appropriate for the intended application. Generally, this entails preparing compositions that are essentially free of pyrogens, as well as other impurities that could be harmful to humans or animals. However, in some embodiments of the present disclosure, a straight rho-inhibiting composition formulation may be administered using one or more of the routes described herein.

In preferred embodiments, the rho-inhibiting compositions of the present disclosure are used in conjunction with appropriate salts and buffers to render delivery of the compositions in a stable manner to allow for uptake by target cells. Buffers also are employed when the rho-inhibiting compositions are introduced into a patient. Aqueous compositions comprise an effective amount of the rho-inhibiting composition to cells dispersed in a pharmaceutically acceptable carrier or aqueous medium. Such compositions also are referred to as inocula. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. Except insofar as any conventional media or agent is incompatible with the vectors or cells of the present disclosure, its use in therapeutic compositions is contemplated. Supplementary active ingredients may also be incorporated into the compositions.

In some embodiments of the present disclosure, the active compositions include classic pharmaceutical preparations. Administration of these compositions according to the present disclosure is via any common route so long as the target tissue is available via that route. This includes oral, nasal, buccal, rectal, vaginal or topical. Alternatively, administration may be by orthotopic, intradermal, subcutaneous, intramuscular, intraperitoneal or intravenous injection.

The active rho-inhibiting compositions of the present disclosure may also be administered parenterally or intraperitoneally or intratumorally. Solutions of the active compounds as free base or pharmacologically acceptable salts are prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it may be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, rho-inhibiting compositions of the present disclosure are administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution is suitably buffered, if necessary, and the liquid diluent first rendered isotonic with sufficient saline or glucose. These particular aqueous solutions are especially suitable for intravenous, intramuscular, subcutaneous and intraperitoneal administration. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). In some embodiments of the present disclosure, the active particles or agents are formulated within a therapeutic mixture to comprise about 0.0001 to 1.0 milligrams, or about 0.001 to 0.1 milligrams, or about 0.1 to 1.0 or even about 10 milligrams per dose or so. Multiple doses may be administered.

Additional formulations that are suitable for other modes of administration include vaginal suppositories and pessaries. A rectal pessary or suppository may also be used.

Suppositories are solid dosage forms of various weights and shapes, usually medicated, for insertion into the rectum, vagina or the urethra. After insertion, suppositories soften, melt or dissolve in the cavity fluids. In general, for suppositories, traditional binders and carriers may include, for example, polyalkylene glycols or triglycerides; such suppositories may be formed from mixtures containing the active ingredient in the range of 0.5% to 10%, preferably 1%-2%. Vaginal suppositories or pessaries are usually globular or oviform and weighing about 5 g each. Vaginal medications are available in a variety of physical forms, e.g., creams, gels or liquids, which depart from the classical concept of suppositories. In addition, suppositories may be used in connection with colon cancer. The rho-inhibiting compositions of the present disclosure also may be formulated as inhalants for the treatment of lung cancer and such like.

### III. Method of Treatment or Prevention of Fibrosis, Cancer, Inflammation, Inflammatory Diseases, and Other Disorders

In some embodiments of the present disclosure, methods and compositions are provided for the treatment of fibrosis, inflammatory disorders, and cancer.

In some embodiments, the compositions and methods described herein find use in the treatment of fibrotic disorders (e.g., systemtic fibrosis or scleroderma).

In some embodiments, the compositions and methods described herein find use in the treatment and/or prevention of Type II diabetes, insulin resistance, megakaryoblastic leukemia, and glioblastoma (See e.g., Jin et al., J Clin Invest. 2011 March; 121(3):918-29; Wiseman et al., J Pediatr Hematol Oncol. 2012 October; 34(7):576-80; Torres et al., Pediatr Blood Cancer. 2011 May; 56(5):846-9; Bernard et al., Med Sci (Paris). 2009 August-September; 25(8-9):676-8; Gilles et al., Blood. 2009 Nov. 5; 114(19):4221-32; Mercher et al., J Clin Invest. 2009 April; 119(4):852-64; Heo et al., Biochem Biophys Res Commun. 2014 Jan. 10; 443(2):749-55; Mertsch et al., Mol Neurobiol. 2013 Oct. 30; Opyrchal et al., Cancer Gene Ther. 2013 November; 20(11):630-7; Fortin Ensign et al., Front Oncol. 2013 Oct. 4; 3:241; each of which is herein incorporated by reference in its entirety) for a discussion of rho signaling in these disorders.

In some embodiments of the present disclosure, methods and compositions are provided for the treatment of inflammatory diseases or inflammatory responses. Inflammation may occur, for example, in response to infection (e.g., infection by a pathogenic organism), wounding, cell damage, or irritants. Inflammatory diseases include but are not limited to arthritis, rheumatoid arthritis, psoriatic arthritis, osteoarthritis, degenerative arthritis, polymyalgia rheumatic, ankylosing spondylitis, reactive arthritis, gout, pseudogout, inflammatory joint disease, systemic lupus erythematosus, polymyositis, and fibromyalgia. Additional types of arthritis include achilles tendinitis, achondroplasia, acromegalic arthropathy, adhesive capsulitis, adult onset Still's disease, anserine bursitis, avascular necrosis, Behcet's syndrome, bicipital tendinitis, Blount's disease, brucellar spondylitis, bursitis, calcaneal bursitis, calcium pyrophosphate deposition disease (CPPD), crystal deposition disease, Caplan's syndrome, carpal tunnel syndrome, chondrocalcinosis, chondromalacia patellae, chronic synovitis, chronic recurrent multifocal osteomyelitis, Churg-Strauss syndrome, Cogan's syndrome, corticosteroid-induced osteoporosis, costosternal syndrome, CREST syndrome, cryoglobulinemia, degenerative joint disease, dermatomyositis, diabetic finger sclerosis, diffuse idiopathic skeletal hyperostosis (DISH), discitis, discoid lupus erythematosus, drug-induced lupus, Duchenne's muscular dystrophy, Dupuytren's contracture, Ehlers-Danlos syndrome, enteropathic arthritis, epicondylitis, erosive inflammatory osteoarthritis, exercise-induced compartment syndrome, Fabry's disease, familial Mediterranean fever, Farber's lipogranulomatosis, Felty's syndrome, Fifth's disease, flat feet, foreign body synovitis, Freiberg's disease, fungal arthritis, Gaucher's disease, giant cell arteritis, gonococcal arthritis, Goodpasture's syndrome, granulomatous arteritis, hemarthrosis, hemochromatosis, Henoch-Schonlein purpura, Hepatitis B surface antigen disease, hip dysplasia, Hurler syndrome, hypermobility syndrome, hypersensitivity vasculitis, hypertrophic osteoarthropathy, immune complex disease, impingement syndrome, Jaccoud's arthropathy, juvenile ankylosing spondylitis, juvenile dermatomyositis, juvenile rheumatoid arthritis, Kawasaki disease, Kienbock's disease, Legg-Calve-Perthes disease, Lesch-Nyhan syndrome, linear scleroderma, lipoid dermatoarthritis, Lofgren's syndrome, Lyme disease, malignant synovioma, Marfan's syndrome, medial plica syndrome, metastatic carcinomatous arthritis, mixed connective tissue disease (MCTD), mixed cryoglobulinemia, mucopolysaccharidosis, multicentric reticulohistiocytosis, multiple epiphyseal dysplasia, mycoplasmal arthritis, myofascial pain syndrome, neonatal lupus, neuropathic arthropathy, nodular panniculitis, ochronosis, olecranon bursitis, Osgood-Schlatter's disease, osteoarthritis, osteochondromatosis, osteogenesis imperfecta, osteomalacia, osteomyelitis, osteonecrosis, osteoporosis, overlap syndrome, pachydermoperiostosis Paget's disease of bone, palindromic rheumatism, patellofemoral pain syndrome, Pellegrini-Stieda syndrome, pigmented villonodular synovitis, piriformis syndrome, plantar fasciitis, polyarteritis nodos, Polymyalgia rheumatic, polymyositis, popliteal cysts, posterior tibial tendinitis, Pott's disease, prepatellar bursitis, prosthetic joint infection, pseudoxanthoma elasticum, psoriatic arthritis, Raynaud's phenomenon, reactive arthritis/Reiter's syndrome, reflex sympathetic dystrophy syndrome, relapsing polychondritis, retrocalcaneal bursitis, rheumatic fever, rheumatoid vasculitis, rotator cuff tendinitis, sacroiliitis, salmonella osteomyelitis, sarcoidosis, saturnine gout, Scheuermann's osteochondritis, scleroderma, septic arthritis, seronegative arthritis, shigella arthritis, shoulder-hand syndrome, sickle cell arthropathy, Sjogren's syndrome, slipped capital femoral epiphysis, spinal stenosis, spondylolysis, staphylococcus arthritis, Stickler syndrome, subacute cutaneous lupus, Sweet's syndrome, Sydenham's chorea, syphilitic arthritis, systemic lupus erythematosus (SLE), Takayasu's arteritis, tarsal tunnel syndrome, tennis elbow, Tietse's syndrome, transient osteoporosis, traumatic arthritis, trochanteric bursitis, tuberculosis arthritis, arthritis of Ulcerative colitis, undifferentiated connective tissue syndrome (UCTS), urticarial vasculitis, viral arthritis, Wegener's granulomatosis, Whipple's disease, Wilson's disease, and yersinial arthritis.

Additional rho-mediated disease states for which compositions and methods of the present disclosure are appropriate include but are not limited to pulmonary arterial hypertension (Naeije et al., Expert Opinin. Pharmacother. 8:2247-2265, 2007; herein incorporated by reference in its entirety); axon regeneration following nerve damage due to spinal cord injury, brain injury, and neurodegenerative diseases (Gross et al., Cell Transpl. 16:245-262, 2007; herein incorporated by reference in its entirety), Raynaud's phenomenon (Flavahan, Rheum. Dis. Clin. North Am. 34:81, 2007; herein incorporated by reference in its entirety), cerebral vascular disease (Chrissobolis et al., Stroke 37:2174-2180, 2006; herein incorporated by reference in its entirety), cardiovascular disease (Noma et al., Am. J Physiol. Cell Physiol. 290(3):C661-8, 2006; herein incorporated by reference in its entirety), and erectile dysfunction (Jin et al., Clin. Sci. (Lond.) 110:153-165, 2006; herein incorporated by reference in its entirety).

In some embodiments, diseases of the lung (e.g., idiopathic pulmonary fibrosis, cystic fibrosis, asthma and COPD, acute respiratory distress syndrome, progressive massive fibrosis a complication of pneumoconiosis and post-lung transplant bronchiolitis obliterans, scleroderma interstitial fibrosis), liver (alcoholic liver cirrhosis, primary biliary cirrhosis, primary sclerosing cholangitis, non-alcoholic steatohepatitis, hepatitis B and C viral disease, idiopathic portal hypertension, autoimmune hepatitis, and drug- and toxicant-induced liver injury), heart (endomyocardial fibrosis, post-myocardial infarction fibrosis, heart failure, atrial fibrosis), skin (scleroderma or systemic sclerosis, eosinic fasciitis, nephrogenic systemic fibrosis or keloid), kidney (diabetic nephropathy, transplant nephropathy, IgA nephropathy, lupus nephritis, and focal sclerosing glomerulonephritis), intestines (inflammatory bowel diseases such as Crohn's disease), eyes (diabetic macular edema, diabetic retinophathy, glaucoma, post-surgical fibrosis, age-related macular degeneration, dry eye) and other fibrotic diseases such as mediastinal fibrosis, retroperitoneal fibrosis, myelofibrosis, Peyronie's disease of the penis, Dupuytren's contracture are treated with the compositions disclosed herein.

It is contemplated that such therapy can be employed in the treatment of any cancer for which a specific signature has been identified or which can be targeted. Cell proliferative disorders, or cancers, contemplated to be treatable with the methods of the present disclosure include human sarcomas and carcinomas, including, but not limited to, fibrosarcoma, myxosarcoma, liposarcoma, chondrosarcoma, osteogenic sarcoma, chordoma, angiosarcoma, endotheliosarcoma, lymphangiosarcoma, Ewing's tumor, lymphangioendotheliosarcoma, synovioma, mesothelioma, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemias, acute lymphocytic leukemia and acute myelocytic leukemia (myeloblastic, promyelocytic, myelomonocytic, monocytic and erythroleukemia), chronic leukemia (chronic myelocytic (granulocytic) leukemia and chronic lymphocytic leukemia), polycythemia vera, lymphoma (Hodgkin's disease and non-Hodgkin's disease), multiple myeloma, Waldenstrom's macroglobulinemia, and heavy chain disease.

“Treating” within the context of the instant disclosure, means an alleviation, in whole or in part, of symptoms associated with a disorder or disease, or slowing, inhibiting or halting of further progression or worsening of those symptoms, or prevention or prophylaxis of the disease or disorder in a subject at risk for developing the disease or disorder. Thus, e.g., treating metastatic cancer may include inhibiting or preventing the metastasis of the cancer, a reduction in the speed and/or number of the metastasis, a reduction in tumor volume of the metastasized cancer, a complete or partial remission of the metastasized cancer or any other therapeutic benefit. As used herein, a “therapeutically effective amount” of a compound of the disclosure refers to an amount of the compound that alleviates, in whole or in part, symptoms associated with a disorder or disease, or slows, inhibits or halts further progression or worsening of those symptoms, or prevents or provides prophylaxis for the disease or disorder in a subject at risk for developing the disease or disorder.

A subject is any animal that can benefit from the administration of a compound as described herein. In some embodiments, the subject is a mammal, for example, a human, a primate, a dog, a cat, a horse, a cow, a pig, a rodent, such as for example a rat or mouse. Typically, the subject is a human.

A therapeutically effective amount of a compound as described herein used in the present disclosure may vary depending upon the route of administration and dosage form. Effective amounts of disclosure compounds typically fall in the range of about 0.001 up to 100 mg/kg/day, and more typically in the range of about 0.05 up to 10 mg/kg/day. Typically, the compound or compounds used in the instant disclosure are selected to provide a formulation that exhibits a high therapeutic index. The therapeutic index is the dose ratio between toxic and therapeutic effects which can be expressed as the ratio between LD₅₀ and ED₅₀. The LD₅₀ is the dose lethal to 50% of the population and the ED₅₀ is the dose therapeutically effective in 50% of the population. The LD₅₀ and ED₅₀ are determined by standard pharmaceutical procedures in animal cell cultures or experimental animals.

The instant disclosure also provides for pharmaceutical compositions and medicaments which may be prepared by combining one or more compounds described herein, pharmaceutically acceptable salts thereof, stereoisomers thereof, tautomers thereof, or solvates thereof, with pharmaceutically acceptable carriers, excipients, binders, diluents or the like to inhibit or treat primary and/or metastatic prostate cancers. Such compositions can be in the form of, for example, granules, powders, tablets, capsules, syrup, suppositories, injections, emulsions, elixirs, suspensions or solutions. The instant compositions can be formulated for various routes of administration, for example, by oral, parenteral, topical, rectal, nasal, or via implanted reservoir. Parenteral or systemic administration includes, but is not limited to, subcutaneous, intravenous, intraperitoneal, and intramuscular injections. The following dosage forms are given by way of example and should not be construed as limiting the instant disclosure.

For oral, buccal, and sublingual administration, powders, suspensions, granules, tablets, pills, capsules, gelcaps, and caplets are acceptable as solid dosage forms. These can be prepared, for example, by mixing one or more compounds of the instant disclosure, or pharmaceutically acceptable salts or tautomers thereof, with at least one additive such as a starch or other additive. Suitable additives are sucrose, lactose, cellulose sugar, mannitol, maltitol, dextran, starch, agar, alginates, chitins, chitosans, pectins, tragacanth gum, gum arabic, gelatins, collagens, casein, albumin, synthetic or semi-synthetic polymers or glycerides. Optionally, oral dosage forms can contain other ingredients to aid in administration, such as an inactive diluent, or lubricants such as magnesium stearate, or preservatives such as paraben or sorbic acid, or antioxidants such as ascorbic acid, tocopherol or cysteine, a disintegrating agent, binders, thickeners, buffers, sweeteners, flavoring agents or perfuming agents. Tablets and pills may be further treated with suitable coating materials known in the art.

Liquid dosage forms for oral administration may be in the form of pharmaceutically acceptable emulsions, syrups, elixirs, suspensions, and solutions, which may contain an inactive diluent, such as water. Pharmaceutical formulations and medicaments may be prepared as liquid suspensions or solutions using a sterile liquid, such as, but not limited to, an oil, water, an alcohol, and combinations of these. Pharmaceutically suitable surfactants, suspending agents, emulsifying agents, may be added for oral or parenteral administration.

As noted above, suspensions may include oils. Such oils include, but are not limited to, peanut oil, sesame oil, cottonseed oil, corn oil and olive oil. Suspension preparations may also contain esters of fatty acids such as ethyl oleate, isopropyl myristate, fatty acid glycerides and acetylated fatty acid glycerides. Suspension formulations may include alcohols, such as, but not limited to, ethanol, isopropyl alcohol, hexadecyl alcohol, glycerol and propylene glycol. Ethers, such as but not limited to, poly(ethyleneglycol), petroleum hydrocarbons such as mineral oil and petrolatum; and water may also be used in suspension formulations.

Injectable dosage forms generally include aqueous suspensions or oil suspensions which may be prepared using a suitable dispersant or wetting agent and a suspending agent. Injectable forms may be in solution phase or in the form of a suspension, which is prepared with a solvent or diluent. Acceptable solvents or vehicles include sterilized water, Ringer's solution, or an isotonic aqueous saline solution. Alternatively, sterile oils may be employed as solvents or suspending agents. Typically, the oil or fatty acid is non-volatile, including natural or synthetic oils, fatty acids, mono-, di- or tri-glycerides.

For injection, the pharmaceutical formulation and/or medicament may be a powder suitable for reconstitution with an appropriate solution as described above. Examples of these include, but are not limited to, freeze dried, rotary dried or spray dried powders, amorphous powders, granules, precipitates, or particulates. For injection, the formulations may optionally contain stabilizers, pH modifiers, surfactants, bioavailability modifiers and combinations of these.

For rectal administration, the pharmaceutical formulations and medicaments may be in the form of a suppository, an ointment, an enema, a tablet or a cream for release of compound in the intestines, sigmoid flexure and/or rectum. Rectal suppositories are prepared by mixing one or more compounds of the instant disclosure, or pharmaceutically acceptable salts or tautomers of the compound, with acceptable vehicles, for example, cocoa butter or polyethylene glycol, which is present in a solid phase at normal storing temperatures, and present in a liquid phase at those temperatures suitable to release a drug inside the body, such as in the rectum. Oils may also be employed in the preparation of formulations of the soft gelatin type and suppositories. Water, saline, aqueous dextrose and related sugar solutions, and glycerols may be employed in the preparation of suspension formulations which may also contain suspending agents such as pectins, carbomers, methyl cellulose, hydroxypropyl cellulose or carboxymethyl cellulose, as well as buffers and preservatives.

Compounds of the disclosure may be administered to the lungs by inhalation through the nose or mouth. Suitable pharmaceutical formulations for inhalation include solutions, sprays, dry powders, or aerosols containing any appropriate solvents and optionally other compounds such as, but not limited to, stabilizers, antimicrobial agents, antioxidants, pH modifiers, surfactants, bioavailability modifiers and combinations of these. Formulations for inhalation administration contain as excipients, for example, lactose, polyoxyethylene-9-lauryl ether, glycocholate and deoxycholate. Aqueous and nonaqueous aerosols are typically used for delivery of inventive compounds by inhalation.

Ordinarily, an aqueous aerosol is made by formulating an aqueous solution or suspension of the compound together with conventional pharmaceutically acceptable carriers and stabilizers. The carriers and stabilizers vary with the requirements of the particular compound, but typically include nonionic surfactants (TWEENs, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions. A nonaqueous suspension (e.g., in a fluorocarbon propellant) can also be used to deliver compounds of the disclosure.

Aerosols containing compounds for use according to the present disclosure are conveniently delivered using an inhaler, atomizer, pressurized pack or a nebulizer and a suitable propellant, e.g., without limitation, pressurized dichlorodifluoromethane, trichlorofluoromethane, dichlorotetrafluoroethane, nitrogen, air, or carbon dioxide. In the case of a pressurized aerosol, the dosage unit may be controlled by providing a valve to deliver a metered amount. Capsules and cartridges of, for example, gelatin for use in an inhaler or insufflator may be formulated containing a powder mix of the compound and a suitable powder base such as lactose or starch.

Delivery of aerosols of the present disclosure using sonic nebulizers is advantageous because nebulizers minimize exposure of the agent to shear, which can result in degradation of the compound.

For nasal administration, the pharmaceutical formulations and medicaments may be a spray, nasal drops or aerosol containing an appropriate solvent(s) and optionally other compounds such as, but not limited to, stabilizers, antimicrobial agents, antioxidants, pH modifiers, surfactants, bioavailability modifiers and combinations of these. For administration in the form of nasal drops, the compounds maybe formulated in oily solutions or as a gel. For administration of nasal aerosol, any suitable propellant may be used including compressed air, nitrogen, carbon dioxide, or a hydrocarbon based low boiling solvent.

Dosage forms for the topical (including buccal and sublingual) or transdermal administration of compounds of the disclosure include powders, sprays, ointments, pastes, creams, lotions, gels, solutions, and patches. The active component may be mixed under sterile conditions with a pharmaceutically-acceptable carrier or excipient, and with any preservatives, or buffers, which may be required. Powders and sprays can be prepared, for example, with excipients such as lactose, talc, silicic acid, aluminum hydroxide, calcium silicates and polyamide powder, or mixtures of these substances. The ointments, pastes, creams and gels may also contain excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Transdermal patches have the added advantage of providing controlled delivery of a compound of the disclosure to the body. Such dosage forms can be made by dissolving or dispersing the agent in the proper medium. Absorption enhancers can also be used to increase the flux of the inventive compound across the skin. The rate of such flux can be controlled by either providing a rate controlling membrane or dispersing the compound in a polymer matrix or gel.

Besides those representative dosage forms described above, pharmaceutically acceptable excipients and carriers are generally known to those skilled in the art and are thus included in the instant disclosure. Such excipients and carriers are described, for example, in “Remingtons Pharmaceutical Sciences” Mack Pub. Co., New Jersey (1991), which is incorporated herein by reference.

The formulations of the disclosure may be designed to be short-acting, fast-releasing, long-acting, and sustained-releasing as described below. Thus, the pharmaceutical formulations may also be formulated for controlled release or for slow release.

The instant compositions may also comprise, for example, micelles or liposomes, or some other encapsulated form, or may be administered in an extended release form to provide a prolonged storage and/or delivery effect. Therefore, the pharmaceutical formulations and medicaments may be compressed into pellets or cylinders and implanted intramuscularly or subcutaneously as depot injections or as implants such as stents. Such implants may employ known inert materials such as silicones and biodegradable polymers.

Specific dosages may be adjusted depending on conditions of disease, the age, body weight, general health conditions, sex, and diet of the subject, dose intervals, administration routes, excretion rate, and combinations of drugs. Any of the above dosage forms containing effective amounts are well within the bounds of routine experimentation and therefore, well within the scope of the instant disclosure.

In some embodiments, the compounds and pharmaceutical compositions described herein are administered in combination with one or more additional agents (e.g., agents useful in the treatment of fibrotic disease, inflammatory disease, or cancer).

Treatments for inflammatory and fibrotic disease include, but are not limited to, nifedipine, amlodipine, diltiazem, felodipine, nicardipine, D-penicillamine, colchicine, PUVA, relaxin, cyclosporine, EPA (omega-3 oil derivative), and immunosuppressive agents (e.g., methotrexate, cyclophosphamide, azathioprine, and mycophenolate).

To kill cells, inhibit cell growth, or metastasis, or angiogenesis, or otherwise reverse or reduce the malignant phenotype of tumor cells using the methods and compositions of the present disclosure in combination therapy, one contacts a “target” cell with the compositions described herein and at least one other agent. These compositions are provided in a combined amount effective to kill or inhibit proliferation of the cell. This process may involve contacting the cells with the immunotherapeutic agent and the agent(s) or factor(s) at the same time. This may be achieved by contacting the cell with a single composition or pharmacological formulation that includes both agents, or by contacting the cell with two distinct compositions or formulations, at the same time.

Alternatively, rho-inhibiting treatment may precede or follow the other agent treatment by intervals ranging from minutes to weeks. In some embodiments it is ensured that a significant period of time did not expire between the time of each delivery, such that the agent and rho-inhibiting composition would still be able to exert an advantageously combined effect on the cell. In such instances, it is contemplated that cells are contacted with both modalities within about 12-24 hours of each other and, more preferably, within about 6-12 hours of each other, with a delay time of only about 12 hours being most preferred. In some situations, it may be desirable to extend the time period for treatment significantly, however, where several days (2 to 7) to several weeks (1 to 8) lapse between the respective administrations.

In some embodiments, more than one administration of the composition of the present disclosure or the other agent is utilized. Various combinations may be employed, where the rho-inhibiting composition is “A” and the other agent is “B”, as exemplified below: A/B/A, B/A/B, B/B/A, A/A/B, B/A/A, A/B/B, B/B/B/A, B/B/A/B, A/A/B/B, A/B/A/B, A/B/B/A, B/B/A/A, B/A/B/A, B/A/A/B, B/B/B/A, A/A/A/B, B/A/A/A, A/B/A/A, A/A/B/A, A/B/B/B, B/A/B/B, B/B/A/B.

In some embodiments of the disclosure, one or more compounds of the disclosure and an additional active agent are administered to a subject, more typically a human, in a sequence and within a time interval such that the compound can act together with the other agent to provide an enhanced benefit relative to the benefits obtained if they were administered otherwise. For example, the additional active agents can be co-administered by co-formulation, administered at the same time or administered sequentially in any order at different points in time; however, if not administered at the same time, they should be administered sufficiently close in time so as to provide the desired therapeutic or prophylactic effect. In some embodiments, the compound and the additional active agents exert their effects at times which overlap. Each additional active agent can be administered separately, in any appropriate form and by any suitable route. In other embodiments, the compound is administered before, concurrently or after administration of the additional active agents.

In various examples, the compound and the additional active agents are administered less than about 1 hour apart, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, no more than 24 hours apart or no more than 48 hours apart. In other examples, the compound and the additional active agents are administered concurrently. In yet other examples, the compound and the additional active agents are administered concurrently by co-formulation.

In other examples, the compound and the additional active agents are administered at about 2 to 4 days apart, at about 4 to 6 days apart, at about 1 week part, at about 1 to 2 weeks apart, or more than 2 weeks apart.

In certain examples, the inventive compound and optionally the additional active agents are cyclically administered to a subject. Cycling therapy involves the administration of a first agent for a period of time, followed by the administration of a second agent and/or third agent for a period of time and repeating this sequential administration. Cycling therapy can provide a variety of benefits, e.g., reduce the development of resistance to one or more of the therapies, avoid or reduce the side effects of one or more of the therapies, and/or improve the efficacy of the treatment.

In other examples, one or more compound of some embodiments of the present disclosure and optionally the additional active agent are administered in a cycle of less than about 3 weeks, about once every two weeks, about once every 10 days or about once every week. One cycle can comprise the administration of an inventive compound and optionally the second active agent by infusion over about 90 minutes every cycle, about 1 hour every cycle, about 45 minutes every cycle, about 30 minutes every cycle or about 15 minutes every cycle. Each cycle can comprise at least 1 week of rest, at least 2 weeks of rest, at least 3 weeks of rest. The number of cycles administered is from about 1 to about 12 cycles, more typically from about 2 to about 10 cycles, and more typically from about 2 to about 8 cycles.

Courses of treatment can be administered concurrently to a subject, i.e., individual doses of the additional active agents are administered separately yet within a time interval such that the inventive compound can work together with the additional active agents. For example, one component can be administered once per week in combination with the other components that can be administered once every two weeks or once every three weeks. In other words, the dosing regimens are carried out concurrently even if the therapeutics are not administered simultaneously or during the same day.

The additional active agents can act additively or, more typically, synergistically with the inventive compound(s). In one example, one or more inventive compound is administered concurrently with one or more second active agents in the same pharmaceutical composition. In another example, one or more inventive compound is administered concurrently with one or more second active agents in separate pharmaceutical compositions. In still another example, one or more inventive compound is administered prior to or subsequent to administration of a second active agent. The disclosure contemplates administration of an inventive compound and a second active agent by the same or different routes of administration, e.g., oral and parenteral. In certain embodiments, when the inventive compound is administered concurrently with a second active agent that potentially produces adverse side effects including, but not limited to, toxicity, the second active agent can advantageously be administered at a dose that falls below the threshold that the adverse side effect is elicited.

Other factors that may be used in combination therapy with the rho-inhibiting compositions of the present disclosure include, but are not limited to, factors that cause DNA damage such as γ-rays, X-rays, and/or the directed delivery of radioisotopes to tumor cells.

Other forms of DNA damaging factors are also contemplated such as microwaves and UV-irradiation. Dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells. The skilled artisan is directed to “Remington's Pharmaceutical Sciences” 15th Edition, chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

In some embodiments of the present disclosure, the regional delivery of rho-inhibiting compositions of some embodiments the present disclosure to patients with cancers is utilized to maximize the therapeutic effectiveness of the delivered agent. Similarly, the chemo- or radiotherapy may be directed to a particular, affected region of the subject's body. Alternatively, systemic delivery of the immunotherapeutic composition and/or the agent may be appropriate in certain circumstances, for example, where extensive metastasis has occurred.

In addition to combining the rho-inhibiting compositions of some embodiments of the present disclosure with chemo- and radiotherapies, it also is contemplated that traditional gene therapies are used. For example, targeting of p53 or p16 mutations along with treatment of the rho-inhibiting compositions of the present disclosure provides an improved anti-cancer treatment. The present disclosure contemplates the co-treatment with other tumor-related genes including, but not limited to, p21, Rb, APC, DCC, NF-I, NF-2, BCRA2, p16, FHIT, WT-I, MEN-I, MEN-II, BRCA1, VHL, FCC, MCC, ras, myc, neu, raf erb, src, fms, jun, trk, ret, gsp, hst, bcl, and abl.

An attractive feature of the present disclosure is that the therapeutic compositions may be delivered to local sites in a patient by a medical device. Medical devices that are suitable for use in the present disclosure include known devices for the localized delivery of therapeutic agents. Such devices include, but are not limited to, catheters such as injection catheters, balloon catheters, double balloon catheters, microporous balloon catheters, channel balloon catheters, infusion catheters, perfusion catheters, etc., which are, for example, coated with the therapeutic agents or through which the agents are administered; needle injection devices such as hypodermic needles and needle injection catheters; needleless injection devices such as jet injectors; coated stents, bifurcated stents, vascular grafts, stent grafts, etc.; and coated vaso-occlusive devices such as wire coils.

Exemplary devices are described in U.S. Pat. Nos. 5,935,114; 5,908,413; 5,792,105; 5,693,014; 5,674,192; 5,876,445; 5,913,894; 5,868,719; 5,851,228; 5,843,089; 5,800,519; 5,800,508; 5,800,391; 5,354,308; 5,755,722; 5,733,303; 5,866,561; 5,857,998; 5,843,003; and 5,933,145; the entire contents of which are incorporated herein by reference. Exemplary stents that are commercially available and may be used in the present application include the RADIUS (SCIMED LIFE SYSTEMS, Inc.), the SYMPHONY (Boston Scientific Corporation), the Wallstent (Schneider Inc.), the PRECEDENT II (Boston Scientific Corporation) and the NIR (Medinol Inc.). Such devices are delivered to and/or implanted at target locations within the body by known techniques.

In some embodiments, composition embodiments of the present disclosure are co-administered with an anti-cancer agent (e.g., chemotherapeutic). In some embodiments, method embodiments of the present disclosure encompass co-administration of an anti-cancer agent (e.g., chemotherapeutic). The present disclosure is not limited by type of anti-cancer agent co-administered. Indeed, a variety of anti-cancer agents are contemplated to be useful in the present disclosure including, but not limited to, Acivicin; Aclarubicin; Acodazole Hydrochloride; Acronine; Adozelesin; Adriamycin; Aldesleukin; Alitretinoin; Allopurinol Sodium; Altretamine; Ambomycin; Ametantrone Acetate; Aminoglutethimide; Amsacrine; Anastrozole; Annonaceous Acetogenins; Anthramycin; Asimicin; Asparaginase; Asperlin; Azacitidine; Azetepa; Azotomycin; Batimastat; Benzodepa; Bexarotene; Bicalutamide; Bisantrene Hydrochloride; Bisnafide Dimesylate; Bizelesin; Bleomycin Sulfate; Brequinar Sodium; Bropirimine; Bullatacin; Busulfan; Cabergoline; Cactinomycin; Calusterone; Caracemide; Carbetimer; Carboplatin; Carmustine; Carubicin Hydrochloride; Carzelesin; Cedefingol; Celecoxib; Chlorambucil; Cirolemycin; Cisplatin; Cladribine; Crisnatol Mesylate; Cyclophosphamide; Cytarabine; Dacarbazine; DACA (N-[2-(Dimethyl-amino)ethyl]acridine-4-carboxamide); Dactinomycin; Daunorubicin Hydrochloride; Daunomycin; Decitabine; Denileukin Diftitox; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Ethiodized Oil I 131; Etoposide; Etoposide Phosphate; Etoprine; Fadrozole Hydrochloride; Fazarabine; Fenretinide; Floxuridine; Fludarabine Phosphate; Fluorouracil; 5-FdUMP; Flurocitabine; Fosquidone; Fostriecin Sodium; FK-317; FK-973; FR-66979; FR-900482; Gemcitabine; Geimcitabine Hydrochloride; Gemtuzumab Ozogamicin; Gold Au 198; Goserelin Acetate; Guanacone; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-1a; Interferon Gamma-1b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Methoxsalen; Metoprine; Meturedepa; Mitindomide; Mitocarcin; Mitocromin; Mitogillin; Mitomalcin; Mitomycin; Mytomycin C; Mitosper; Mitotane; Mitoxantrone Hydrochloride; Mycophenolic Acid; Nocodazole; Nogalamycin; Oprelvekin; Ormaplatin; Oxisuran; Paclitaxel; Pamidronate Disodium; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rituximab; Rogletimide; Rolliniastatin; Safingol; Safingol Hydrochloride; Samarium/Lexidronam; Semustine; Simtrazene; Sparfosate Sodium; Sparsomycin; Spirogermanium Hydrochloride; Spiromustine; Spiroplatin; Squamocin; Squamotacin; Streptonigrin; Streptozocin; Strontium Chloride Sr 89; Sulofenur; Talisomycin; Taxane; Taxoid; Tecogalan Sodium; Tegafur; Teloxantrone Hydrochloride; Temoporfin; Teniposide; Teroxirone; Testolactone; Thiamiprine; Thioguanine; Thiotepa; Thymitaq; Tiazofurin; Tirapazamine; Tomudex; TOP-53; Topotecan Hydrochloride; Toremifene Citrate; Trastuzumab; Trestolone Acetate; Triciribine Phosphate; Trimetrexate; Trimetrexate Glucuronate; Triptorelin; Tubulozole Hydrochloride; Uracil Mustard; Uredepa; Valrubicin; Vapreotide; Verteporfin; Vinblastine; Vinblastine Sulfate; Vincristine; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride; 2-Chlorodeoxyadenosine; 2′-Deoxyformycin; 9-aminocamptothecin; raltitrexed; N-propargyl-5,8-dideazafolic acid; 2-chloro-2′-arabino-fluoro-2′-deoxyadenosine; 2-chloro-2′-deoxyadenosine; anisomycin; trichostatin A; hPRL-G129R; CEP-751; linomide; sulfur mustard; nitrogen mustard (mechlorethamine); cyclophosphamide; melphalan; chlorambucil; ifosfamide; busulfan; N-methyl-N-nitrosourea (MNU); N, N′-Bis(2-chloroethyl)-N-nitrosourea (BCNU); N-(2-chloroethyl)-N′-cyclohex-yl-N-nitrosourea (CCNU); N-(2-chloroethyl)-N′-(trans-4-methylcyclohexyl-N— nitrosourea (MeCCNU); N-(2-chloroethyl)-N′-(diethyl)ethylphosphonate-N-nit-rosourea (fotemustine); streptozotocin; diacarbazine (DTIC); mitozolomide; temozolomide; thiotepa; mitomycin C; AZQ; adozelesin; Cisplatin; Carboplatin; Ormaplatin; Oxaliplatin; C1-973; DWA 2114R; JM216; JM335; Bis (platinum); tomudex; azacitidine; cytarabine; gemcitabine; 6-Mercaptopurine; 6-Thioguanine; Hypoxanthine; teniposide; 9-amino camptothecin; Topotecan; CPT-11; Doxorubicin; Daunomycin; Epirubicin; darubicin; mitoxantrone; losoxantrone; Dactinomycin (Actinomycin D); amsacrine; pyrazoloacridine; all-trans retinol; 14-hydroxy-retro-retinol; all-trans retinoic acid; N-(4-Hydroxyphenyl) retinamide; 13-cis retinoic acid; 3-Methyl TTNEB; 9-cis retinoic acid; fludarabine (2-F-ara-AMP); and 2-chlorodeoxyadenosine (2-Cda).

Other anti-cancer agents include: Antiproliferative agents (e.g., Piritrexim Isothionate), Antiprostatic hypertrophy agent (e.g., Sitogluside), Benign prostatic hypertrophy therapy agents (e.g., Tamsulosin Hydrochloride), Prostate growth inhibitor agents (e.g., Pentomone), and Radioactive agents: Fibrinogen I 125; Fludeoxyglucose F 18; Fluorodopa F 18; Insulin I 125; Insulin I 131; Iobenguane I 123; Iodipamide Sodium I 131; Iodoantipyrine I 131; Iodocholesterol I 131; Iodohippurate Sodium I 123; Iodohippurate Sodium I 125; Iodohippurate Sodium I 131; Iodopyracet I 125; Iodopyracet I 131; Iofetamine Hydrochloride I 123; Iomethin I 125; Iomethin I 131; Iothalamate Sodium I 125; Iothalamate Sodium 1131; Iotyrosine 1131; Liothyronine 1125; Liothyronine 1131; Merisoprol Acetate Hg 197; Merisoprol Acetate Hg 203; Merisoprol Hg 197; Selenomethionine Se 75; Technetium Tc 99m Antimony Trisulfide Colloid; Technetium Tc 99m Bicisate; Technetium Tc 99m Disofenin; Technetium Tc 99m Etidronate; Technetium Tc 99m Exametazime; Technetium Tc 99m Furifosmin; Technetium Tc 99m Gluceptate; Technetium Tc 99m Lidofenin; Technetium Tc 99m Mebrofenin; Technetium Tc 99m Medronate; Technetium Tc 99m Medronate Disodium; Technetium Tc 99m Mertiatide; Technetium Tc 99m Oxidronate; Technetium Tc 99m Pentetate; Technetium Tc 99m Pentetate Calcium Trisodium; Technetium Tc 99m Sestamibi; Technetium Tc 99m Siboroxime; Technetium Tc 99m Succimer; Technetium Tc 99m Sulfur Colloid; Technetium Tc 99m Teboroxime; Technetium Tc 99m Tetrofosmin; Technetium Tc 99m Tiatide; Thyroxine I 125; Thyroxine I 131; Tolpovidone 1131; Triolein 1125; Triolein 1131.

Another category of anti-cancer agents is anti-cancer Supplementary Potentiating Agents, including: Tricyclic anti-depressant drugs (e.g., imipramine, desipramine, amitryptyline, clomipramine, trimipramine, doxepin, nortriptyline, protriptyline, amoxapine and maprotiline); non-tricyclic anti-depressant drugs (e.g., sertraline, trazodone and citalopram); Ca⁺⁺ antagonists (e.g., verapamil, nifedipine, nitrendipine and caroverine); Calmodulin inhibitors (e.g., prenylamine, trifluoroperazine and clomipramine); Amphotericin B; Triparanol analogues (e.g., tamoxifen); antiarrhythmic drugs (e.g., quinidine); antihypertensive drugs (e.g., reserpine); Thiol depleters (e.g., buthionine and sulfoximine) and Multiple Drug Resistance reducing agents such as Cremaphor EL.

Still other anticancer agents are those selected from the group consisting of: annonaceous acetogenins; asimicin; rolliniastatin; guanacone, squamocin, bullatacin; squamotacin; taxanes; paclitaxel; gemcitabine; methotrexate FR-900482; FK-973; FR-66979; FK-317; 5-FU; FUDR; FdUMP; Hydroxyurea; Docetaxel; discodermolide; epothilones; vincristine; vinblastine; vinorelbine; meta-pac; irinotecan; SN-38; 10-OH campto; topotecan; etoposide; adriamycin; flavopiridol; Cis-Pt; carbo-Pt; bleomycin; mitomycin C; mithramycin; capecitabine; cytarabine; 2-C1-2′deoxyadenosine; Fludarabine-PO₄; mitoxantrone; mitozolomide; Pentostatin; and Tomudex.

One particularly preferred class of anticancer agents are taxanes (e.g., paclitaxel and docetaxel). Another important category of anticancer agent is annonaceous acetogenin.

Other cancer therapies include hormonal manipulation. In some embodiments, the anti-cancer agent is tamoxifen or the aromatase inhibitor arimidex (i.e., anastrozole).

In some embodiments of the present disclosure, to gain a general perspective into the safety of a particular rho-inhibiting composition of an embodiment of the present disclosure, toxicity testing is performed. Toxicological information may be derived from numerous sources including, but not limited to, historical databases, in vitro testing, and in vivo animal studies.

In vitro toxicological methods have gained popularity in recent years due to increasing desires for alternatives to animal experimentation and an increased perception to the potential ethical, commercial, and scientific value. In vitro toxicity testing systems have numerous advantages including improved efficiency, reduced cost, and reduced variability between experiments. These systems also reduce animal usage, eliminate confounding systemic effects (e.g., immunity), and control environmental conditions.

Although any in vitro testing system may be used with the present disclosure, the most common approach utilized for in vitro examination is the use of cultured cell models. These systems include freshly isolated cells, primary cells, or transformed cell cultures. Cell culture as the primary means of studying in vitro toxicology is advantageous due to rapid screening of multiple cultures, usefulness in identifying and assessing toxic effects at the cellular, subcellular, or molecular level. In vitro cell culture methods commonly indicate basic cellular toxicity through measurement of membrane integrity, metabolic activities, and subcellular perturbations. Commonly used indicators for membrane integrity include cell viability (cell count), clonal expansion tests, trypan blue exclusion, intracellular enzyme release (e.g. lactate dehydrogenase), membrane permeability of small ions (K⁺, Ca²⁺), and intracellular Ala accumulation of small molecules (e.g., ⁵¹Cr, succinate). Subcellular perturbations include monitoring mitochondrial enzyme activity levels via, for example, the MTT test, the WST1 assay, determining cellular adenine triphosphate (ATP) levels, neutral red uptake into lysosomes, and quantification of total protein synthesis. Metabolic activity indicators include glutathione content, lipid peroxidation, and lactate/pyruvate ratio. It should be noted that compounds having toxicity may still be employed in appropriate circumstances, e.g., for research use.

The MTT assay is a fast, accurate, and reliable methodology for obtaining cell viability measurements. The MTT assay was first developed by Mosmann (See, e.g., Mosmann, J. Immunol. Meth., 65:55 (1983)). It is a simple colorimetric assay numerous laboratories have utilized for obtaining toxicity results (See e.g., Kuhlmann et al., Arch. Toxicol., 72:536 (1998)). Briefly, the mitochondria produce ATP to provide sufficient energy for the cell. In order to do this, the mitochondria metabolize pyruvate to produce acetyl CoA. Within the mitochondria, acetyl CoA reacts with various enzymes in the tricarboxylic acid cycle resulting in subsequent production of ATP. One of the enzymes particularly useful in the MTT assay is succinate dehydrogenase. MTT (3-(4,5-dimethylthiazol-2-yi)-2 diphenyl tetrazolium bromide) is a yellow substrate that is cleaved by succinate dehydrogenase forming a purple formazan product. The alteration in pigment identifies changes in mitochondria function. Nonviable cells are unable to produce formazan, and therefore, the amount produced directly correlates to the quantity of viable cells. Absorbance at 540 nm is utilized to measure the amount of formazan product.

An alternative to the MTT assay is the WST-1 assay, which similarly is based on measurement of metabolic activity to measure toxin effects on mammalian cells but uses a different substrate, 4-[3-(4-iodophenyl)-2-(4-nitrophenyl)-2H-5-tetrazolio]-1,3-benzene disulfonate (Dietrich et al., Appl. Environ. Microbiol., 65:4470 (1999); Kau et al., Curr. Microbiol., 44:106 (2002); Scobie et al., PNAS, 100:5170 (2003); Moravek et al., FEMS Microbiol. Lett., 257:293 (2006); Ngamwongsatit et al., J. Microbiol. Methods, 73:211 (2008); each herein incorporated by reference in its entirety). In the WST-1 assay, mitochondrial succinate-tetrazolium reductase reacts with the WST-1 reagent to produce water-soluble formazan dye. This water solubility is an advantage over the classical MTT assay, as the product of the WST-1 assay can be quantified in 0.4-4 h without additional solubilization steps (Ngamwongsatit et al., J. Microbiol. Methods, 73:211 (2008); herein incorporated by reference in its entirety). Therefore, in some cases, WST-1 assays may be use preferentially to MTT assays if handling time is a concern (e.g., in high-throughput screens).

The results of the in vitro tests can be compared to in vivo toxicity tests in order to extrapolate to live animal conditions. Typically, acute toxicity from a single dose of the substance is assessed. Animals are monitored over 14 days for any signs of toxicity (increased temperature, breathing difficulty, death, etc). Traditionally, the standard of acute toxicity is the median lethal dose (LD₅₀), which is the predicted dose at which half of the treated population would be killed. The determination of this dose occurs by exposing test animals to a geometric series of doses under controlled conditions. Other tests include subacute toxicity testing, which measures the animal's response to repeated doses of the composition for no longer than 14 days. Subchronic toxicity testing involves testing of a repeated dose for 90 days. Chronic toxicity testing is similar to subchronic testing but may last for over a 90-day period. In vivo testing can also be conducted to determine toxicity with respect to certain tissues. For example, in some embodiments of the present disclosure, tumor toxicity (e.g., effect of the compositions of the present disclosure on the survival of tumor tissue) is determined (e.g., by detecting changes in the size and/or growth of tumor cells or tissues).

### EXAMPLES

The following examples are provided in order to demonstrate and further illustrate certain preferred embodiments and aspects of the present disclosure and are not to be construed as limiting the scope thereof.

### Example 1

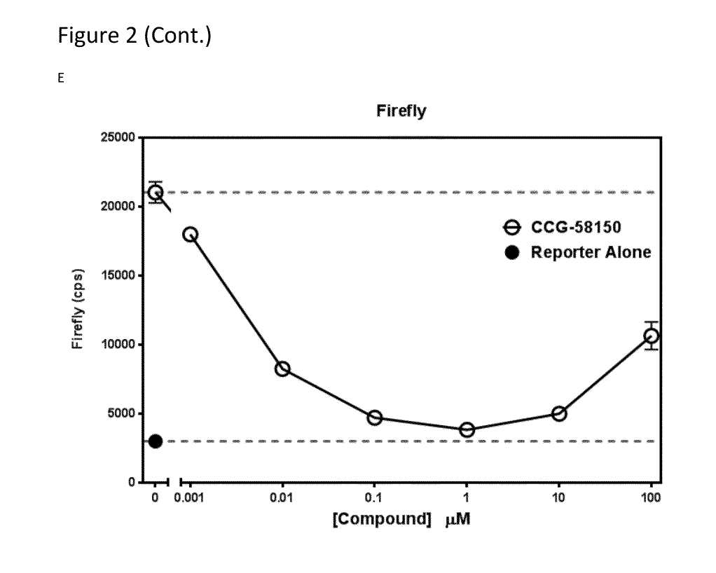

CCG-1423 was a cellular IC₅₀ of ˜1 uM to inhibit SRE-Luciferase expression (Evelyn, C. R., Wade, S. M., Wang, Q., Wu, M., Iniguez-Lluhi, J. A., Merajver, S. D., and Neubig, R. R. 2007. CCG-1423: a small-molecule inhibitor of RhoA transcriptional signaling. Mol Cancer Ther 6:2249-2260). It has been used in many labs as a tool compound for blocking MRTF/SRF-regulated gene transcription (Sandbo et al., et al., 2011. Am J Physiol Lung Cell Mol Physiol 301:L656-666; Sandbo, et al., 2009. Am J Respir Cell Mol Biol 41:332-338; Evelyn, et al., 2007. Mol Cancer Ther 6:2249-2260; Sandbo et al., 2013. J Biol Chem 288:15466-15473; Prencipe et al., 2013. Prostate 73:743-753; Sakai et al., 2013. FASEB J 27:1830-1846; Buller et al., 2012. Glia 60:1906-1914; Chong et al., 2012. PLoS One 7:e40966; Jin et al., 2011. J Clin Invest 121:918-929; Mae et al., 2010. Biochem Biophys Res Commun 393:877-882; Evelyn et al., 2010. Bioorg Med Chem Lett 20:665-672; Lu et al., 2009. Curr Med Chem 16:1355-1365). CCG-1423 acts by preventing nuclear accumulation of MRTF (Evelyn et al, supra; Jin et al., supra). CCG-203971 produces less acute cellular toxicity (FIG. 2B) and is much better tolerated in vivo. A second series (CCG-58146) was found in a follow-up HTS campaign and exhibits much higher potency (nM IC₅₀ in SRE-luciferase (FIG. 2C). This series acts by a somewhat different mechanism; it doesn't block MRTF nuclear localization but does effectively inhibit gene transcription.

Both compounds are more effective than ROCK inhibitors at reducing SRF-mediated transcription (FIG. 2D & Evelyn et al, spura). Several groups have used CCG-1423 to interdict myofibroblast formation in vitro (Zhou, et al., 2013. J Clin Invest 123:1096-1108; Sandbo et al., 2011. Am J Physiol Lung Cell Mol Physiol 301:L656-666; Sandbo et al., 2009. Am J Respir Cell Mol Biol 41:332-338) and it was recently shown to have in vivo activity in a bleomycin model of peritoneal fibrosis (Sakai et al., 2013. FASEB J.). CCG-203971, a MRTF/SRF-gene transcription inhibitor, reverses the myofibroblast phenotype in vitro for both TGFb-stimulated normal human, dermal fibroblasts as well as the spontaneous activation of fibroblasts derived from scleroderma patients (SSc). Furthermore, it prevented fibrosis in a mouse bleomycin skin injury model.

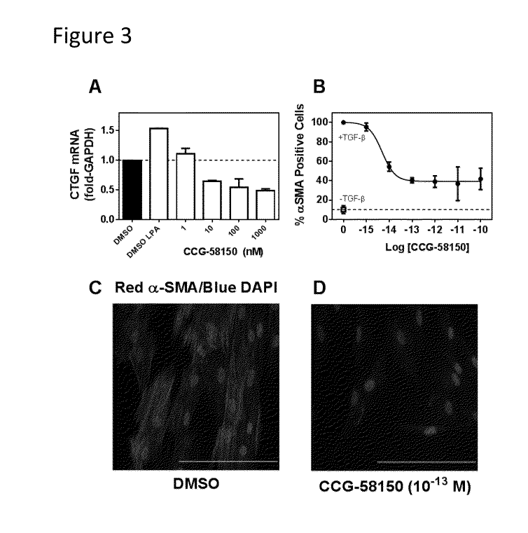

A second series, based on CCG-58146, has very high potency, metabolic stability, and ability to reverse myofibroblast differentiation in vitro. It selectively blocks MRTF/SRF-regulated gene transcription without preventing MRTF nuclear localization. A close analog, CCG-58150, inhibits expression of endogenous CTGF mRNA with a 1 nM IC₅₀ (FIG. 3) and blocks myofibroblast differentiation at pM concentrations.

Disruption of the Rho pathway with inhibitors of the Rho associated, coiled-coil containing protein kinase (ROCK) has reversed myofibroblast differentiation in vitro and fibrosis in several animal models (Akhmetshina et al., Arthritis Rheum 58: 2553-2564, 2008; Buhl et al., J Biol Chem 270: 24631-24634, 1995; Masszi et al., Am J Physiol Renal Physiol 284: F911-924, 2003; Sandbo et al., Am J Physiol Lung Cell Mol Physiol 301: L656-666, 2011; J Cardiovasc Transl Res 5: 794-804, 2012; Zhao et al., J Cell Sci 120: 1801-1809, 2007; Zhou et al., J Clin Invest 123: 1096-1108, 2013). CCG-1423, which blocks MRTF nuclear localization downstream of ROCK and disrupts SRF-mediated gene transcription (Evelyn et al., Mol Cancer Ther 6: 2249-2260, 2007) is more effective than ROCK inhibitors in reducing SRF-mediated transcription (Evelyn et al., Mol Cancer Ther 6: 2249-2260, 2007). Several groups have used this compound to interdict myofibroblast formation (Sandbo et al., Am J Respir Cell Mol Biol 41: 332-338, 2009; Zhou et al., supra) and it was recently shown to have in vivo activity in a chlorhexidine gluconate model of peritoneal fibrosis (Sakai et al., supra). The CCG-203971 series has been optimized to reduce off-target toxicity (Bell et al., Bioorg Med Chem Lett 23: 3826-3832, 2013; Evelyn et al., Bioorg Med Chem Lett 20: 665-672, 2010).

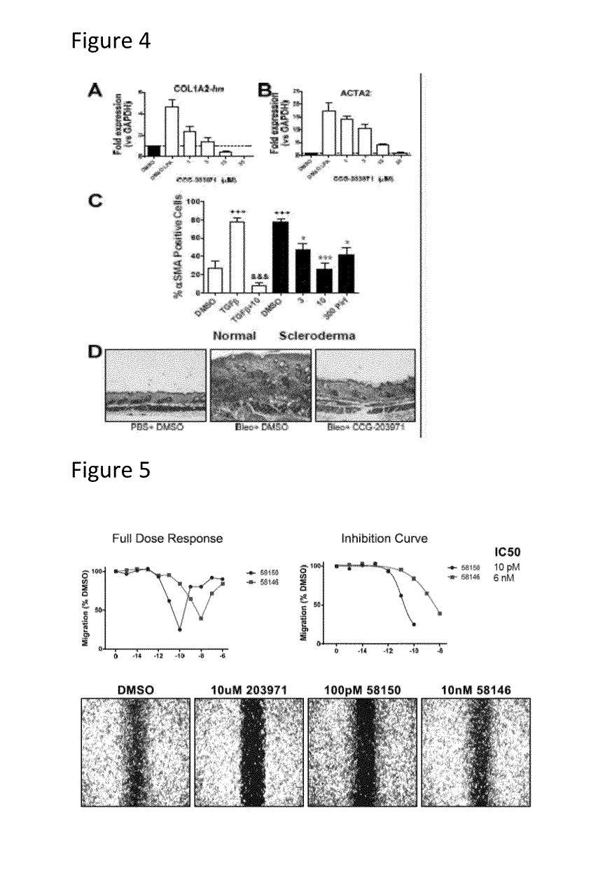

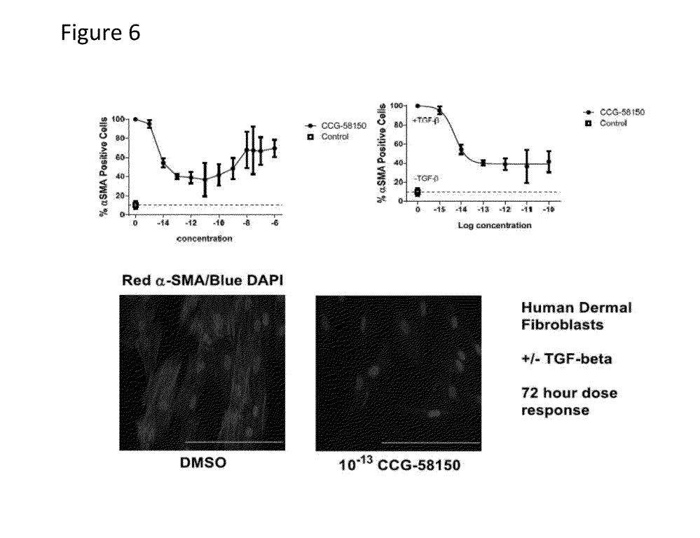

A recent study of human SSc dermal fibroblasts showed pronounced in vitro inhibition of fibrosis markers (COL1A2-hn and ACTA2 mRNA) as well as a-SMA protein expression (FIG. 4). In FIG. 4C, CCG-203971 is 100× more potent than perfenidone, the only antifibrotic drug approved by a national agency (Japan and Europe). CCG-203971 also prevented fibrosis in a bleomycin skin injury model (100 mg/kg b.i.d.). It has poor pharmacokinetics—low oral absorption and rapid hepatic clearance.

A new compound series (CCG-518150) that is much more potent in vitro (FIG. 2) has been identified. It has nM potency in a 24-hour assay in NIH 3T3 cells and fM-pM potency in a 72-hour assay in human primary dermal fibroblasts. Importantly, it has highly favorable stability in liver microsomes and very good in vivo pharmacokinetics. Blood levels 24 hours after a single oral dose exceeded the in vitro IC50 by over 10-fold.

### Example 2

This example describes experiments that assess the tolerability and activity of CCG-51850 in vivo.

The experiments shown in FIG. 4 are repeated CCG-58150 and derivatives thereof. A range of doses (0.1, 1, and 10 mg/kg) is used to increase the likelihood of finding a well-tolerated dose that is effective. Preliminary tolerability studies indicated that 10 mg/kg i.p. daily for 2-weeks was tolerated in normal mice.

Protocol:

Twelve week old male C57bl/6 mice, weighing approximately 25 g, are acclimated to the laboratory environment for at least 1 week then randomized into 5 experimental groups.

Treatment Schedule:

| | | | Treatment 1 | | Treatment 2 | |
|---|---|---|---|---|---|---|
| | | Animal | Bleomycin | | CCG-58150 | |
| Group | Sex | No. | mg | Day | mg/kg | Day |
| 1 | M | 1-10 | Vehicle 1 | 1-14 | Vehicle 2 | 1-14 |
| 2 | M | 11-20 | 0.1 mg | 1-14 | Vehicle 2 | 1-14 |
| 3 | M | 21-30 | 0.1 mg | 1-14 | 0.1 | 1-14 |
| 4 | M | 31-40 | 0.1 mg | 1-14 | 1 | 1-14 |
| 5 | M | 41-50 | 0.1 mg | 1-14 | 10 | 1-14 |

Vehicle 1: Sterile Phosphate Buffered Saline (PBS)
Vehicle 2: 20% DMSO/30% PEG/50% PBS

Mice are weighed once prior to study start. Dermal fibrosis is induced in Groups 2-5 by daily intradermal injections of bleomycin over 14 days. Group 1 will receive vehicle 1 (PBS) over the same days. Daily therapeutic dosing with either vehicle 2 or CCG-58150 by oral gavage coincide with the intradermal injection study days. Mice are anesthetized using isoflurane during intradermal injections. The back is shaved and the injection site (˜1 cm2) cleaned with three alternating passes of chlorhexidine and warm, sterile saline or water. The periphery of the injection site is circled using a sharpie marker as a visual aid.

Bleomycin is dissolved in sterile PBS at a concentration of 1 mg/ml, sterile filtered, and frozen in aliquots appropriately sized for the daily injections prior to the start of the dosing period. 100 μl of bleomycin or PBS (Vehicle 1) is administered to anesthetized mice by intradermal injection using a 0.5 cc TB syringe with a 27 G needle. Following bleomycin or vehicle administration, test substance (CCG-58150) or vehicle 2 (20% DMSO/30% PEG/50% PBS) is administered by oral gavage at 10 μL/gram of body weight. Mice receive daily clinical observations, injections of vehicle/bleomycin and oral control/test substance for 14 days.

Mice are euthanized on Day 15. A terminal blood sample is collected for determination of serum chemistry values. The liver is collected and fixed in neutral buffered formalin, slides prepared and stained with Hematoxolin and eosin (H&E). The skin at the injection site is excised with scissors to contain a few mm of normal skin around the perimeter of the fibrotic skin. A small section (˜5×2 mm) of the fibrotic skin is placed in a microfuge tube, snap frozen in liquid nitrogen, and stored at −80° C. for measurement of hydroxyproline content and mRNA for CTGF, ACTA2, and CollA2-hn by RT-PCR. The remaining tissue is pinned flat and fixed in 10% buffered formalin at room temperature. After fixation for at least 24 hours, the skin is cut into 1-2 mm wide strips and embedded in paraffin with the tissue cross section facing up and all strips oriented in the same direction in the cassette. Slides are prepared and stained with H& E and Masson's trichrome. Images are collected on an Evos microscope and skin thickness and intensity of fibrosis of two distinct images from each animal is analyzed using ImageJ. Statistical analysis is done with GraphPad 6.0 software. The significance of the bleomycin effect is done using an unpaired t-test for control vs bleomycin. Compound effects are analyzed using 1-way ANOVA of all bleomycin-treated mice with doses of compound (0, 0.1, 1, 10 mg/kg) as the independent variable. A p value <0.05 is considered statistically significant.

Marked increases in skin thickness, hydroxyl-proline content, and fibrotic biomarkers (CTGF, ACTA2, hn-COL1A2 mRNA) are expected with bleomycin injections as seen previously (FIG. 4D). A dose dependent reduction in those parameters with compound treatment is expected. In vitro effects of CCG-58150 to reduce alpha-SMA staining (FIG. 2B) are much more potent (fM-pM) than those seen for the earlier chemical series (FIG. 4C). However, the maximum suppression with CCG-58150 is slightly less (60% vs 80% inhibition of % αSMA positive cells). A full or partial suppression of fibrosis by the CCG-58150 series is expected. Given the very strongly significant effect of CCG-203971 (p<0.001) for reduction of skin fibrosis and the larger group sizes in the present study (n=10 vs n=7 previously), it is anticipated that a partial decrease in skin fibrosis can be detected.

### Example 3

Compounds were prepared as exemplified in Scheme A or B.

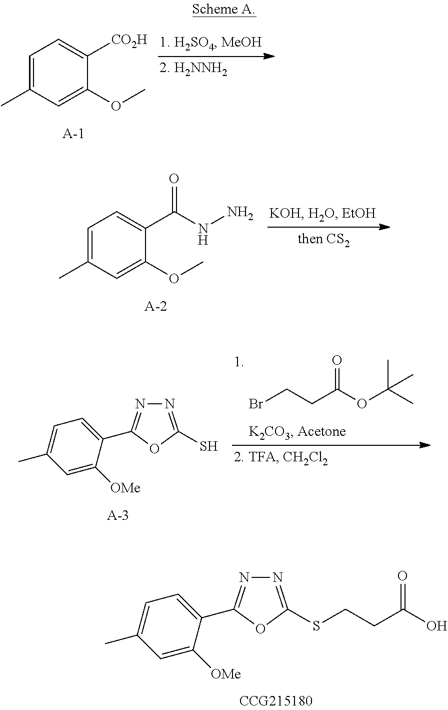

2-Methoxy-4-methylbenzoic acid A-1 was converted to hydrazide A-2 by the method reported in the literature (Bioorg. Med Chem Lett 2011, 19, 5031). Cyclization to 2-mercapto-1,3,4-oxadiazole A-3 was effected with sodium hydroxide and carbon disulfide (J. Am. Chem. Soc. 1955, 77, 400). S-alkylation with t-butyl 3-bromopropionate under basic conditions followed by acidic hydrolysis of the t-butyl ester provided 215180.

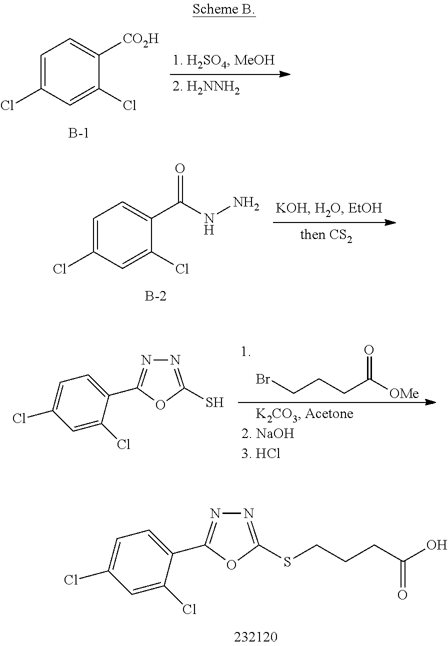

TABLE 2
Rho inhibition by a variety of compounds

| | SRE-Luciferase | | |
|---|---|---|---|
| ID | IC50 (uM) | Source | Structure |
| 20321 | >100 | ChemDiv (San Diego, CA) | 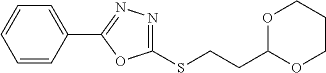 [SMILES: c1ccc(-c2nnc(SCCC3OCCCO3)o2)cc1] |
| 58146 | 0.18 | ChemDiv (San Diego, CA) | 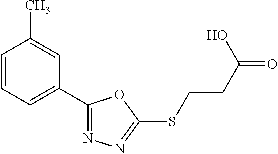 [SMILES: Cc1cccc(-c2nnc(SCCC(=O)O)o2)c1] |
| 58150 | 0.004 | ChemDiv (San Diego, CA) | 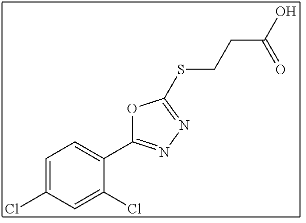 [SMILES: O=C(O)CCSc1nnc(-c2ccc(Cl)cc2Cl)o1] |
| 105557 | 5.2 | ChemDiv (San Diego, CA) | 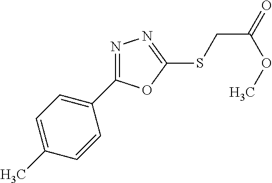 [SMILES: COC(=O)CSc1nnc(-c2ccc(C)cc2)o1] |
| 107983 | >100 | ChemDiv (San Diego, CA) | 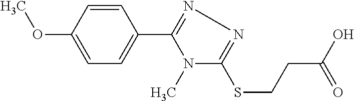 [SMILES: COc1ccc(-c2nnc(SCCC(=O)O)n2C)cc1] |
| 111085 | >100 | ChemDiv (San Diego, CA) | 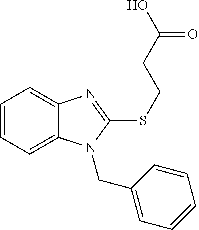 [SMILES: O=C(O)CCSc1nc2ccccc2n1Cc1ccccc1] |
| 114898 | 6.7 | ChemDiv (San Diego, CA) | 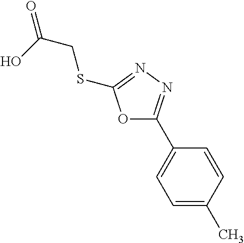 [SMILES: Cc1ccc(-c2nnc(SCC(=O)O)o2)cc1] |
| 123851 | 1.5 | ChemDiv (San Diego, CA) | 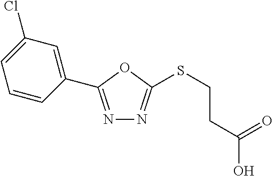 [SMILES: O=C(O)CCSc1nnc(-c2cccc(Cl)c2)o1] |
| 123852 | 0.34 | ChemDiv (San Diego, CA) | 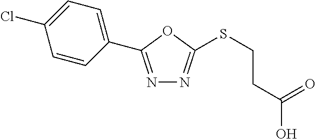 [SMILES: O=C(O)CCSc1nnc(-c2ccc(Cl)cc2)o1] |
| 123853 | 6.4 | ChemDiv (San Diego, CA) | 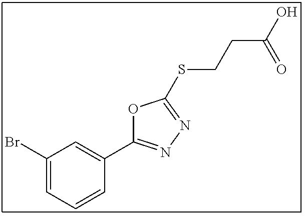 [SMILES: O=C(O)CCSc1nnc(-c2cccc(Br)c2)o1] |
| 123855 | 0.16 | ChemDiv (San Diego, CA) | 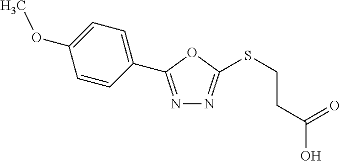 [SMILES: COc1ccc(-c2nnc(SCCC(=O)O)o2)cc1] |
| 123859 | 3.5 | ChemDiv (San Diego, CA) | 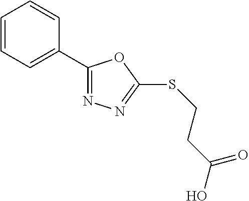 [SMILES: O=C(O)CCSc1nnc(-c2ccccc2)o1] |
| 123860 | 43 | ChemDiv (San Diego, CA) | 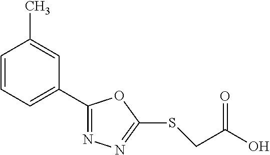 [SMILES: Cc1cccc(-c2nnc(SCC(=O)O)o2)c1] |
| 123862 | 0.034 | ChemDiv (San Diego, CA) | 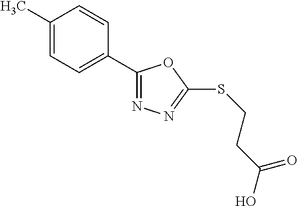 [SMILES: Cc1ccc(-c2nnc(SCCC(=O)O)o2)cc1] |
| 123867 | >100 | ChemDiv (San Diego, CA) | 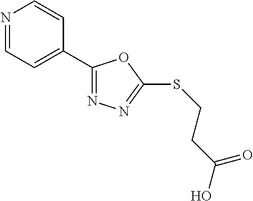 [SMILES: O=C(O)CCSc1nnc(-c2ccncc2)o1] |
| 123869 | 87 | ChemDiv (San Diego, CA) | 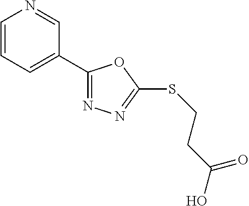 [SMILES: O=C(O)CCSc1nnc(-c2cccnc2)o1] |
| 123873 | >100 | ChemDiv (San Diego, CA) | 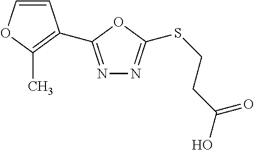 [SMILES: Cc1occc1-c1nnc(SCCC(=O)O)o1] |
| 211911 | 3.6 | ChemDiv (San Diego, CA) | 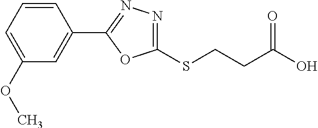 [SMILES: COc1cccc(-c2nnc(SCCC(=O)O)o2)c1] |
| 211912 | >100 | ChemDiv (San Diego, CA) | 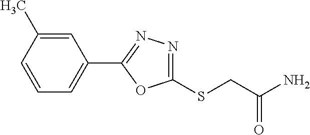 [SMILES: Cc1cccc(-c2nnc(SCC(N)=O)o2)c1] |
| 211913 | >100 | ChemDiv (San Diego, CA) | 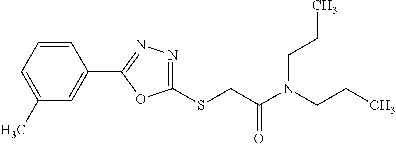 [SMILES: CCCN(CCC)C(=O)CSc1nnc(-c2cccc(C)c2)o1] |
| 211914 | >100 | ChemDiv (San Diego, CA) | 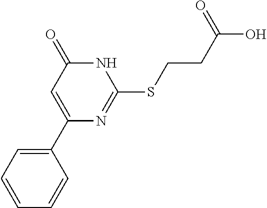 [SMILES: O=C(O)CCSc1nc(-c2ccccc2)cc(=O)[nH]1] |
| 215027 | >100 | ChemDiv (San Diego, CA) | 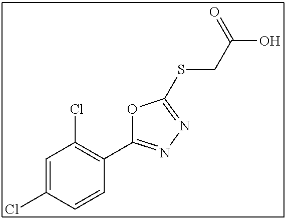 [SMILES: O=C(O)CSc1nnc(-c2ccc(Cl)cc2Cl)o1] |
| 215028 | >100 | ChemDiv (San Diego, CA) | 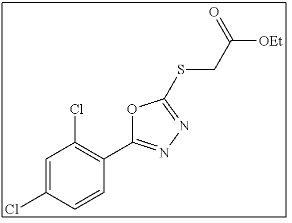 [SMILES: CCOC(=O)CSc1nnc(-c2ccc(Cl)cc2Cl)o1] |
| 215029 | >100 | ChemDiv (San Diego, CA) | 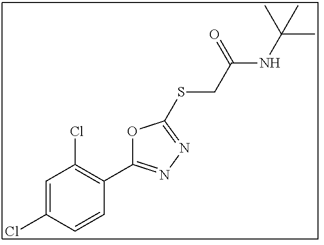 [SMILES: CC(C)(C)NC(=O)CSc1nnc(-c2ccc(Cl)cc2Cl)o1] |
| 215104 | >100 | Ryan Scientific (Mount Pleasant, SC) | 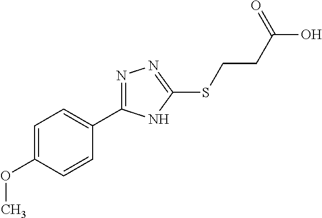 [SMILES: COc1ccc(-c2nnc(SCCC(=O)O)[nH]2)cc1] |
| 215160 | >100 | Synthesis Scheme A | 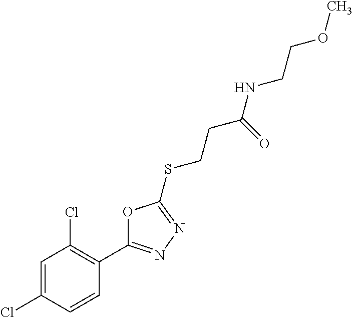 [SMILES: COCCNC(=O)CCSc1nnc(-c2ccc(Cl)cc2Cl)o1] |
| 215161 | 0.0026 | Synthesis Scheme A | 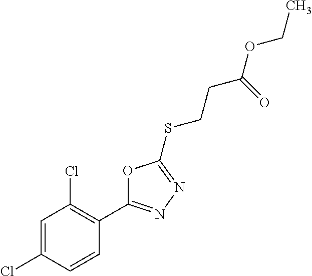 [SMILES: CCOC(=O)CCSc1nnc(-c2ccc(Cl)cc2Cl)o1] |
| 215180 | 0.038 | Synthesis Scheme A | 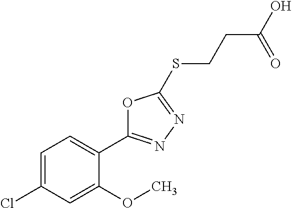 [SMILES: COc1cc(Cl)ccc1-c1nnc(SCCC(=O)O)o1] |
| 215201 | 0.023 | Synthesis Scheme A | 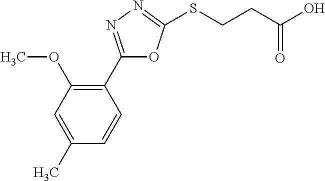 [SMILES: COc1cc(C)ccc1-c1nnc(SCCC(=O)O)o1] |
| 215220 | 0.0026 | Synthesis Scheme A | 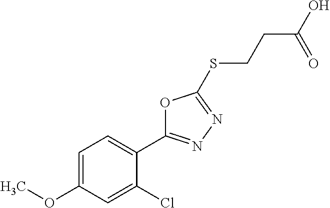 [SMILES: COc1ccc(-c2nnc(SCCC(=O)O)o2)c(Cl)c1] |
| 215240 | 24 | Synthesis Scheme A | 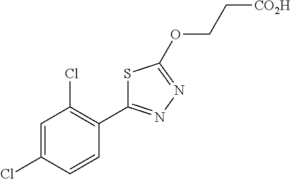 [SMILES: O=C(O)CCOc1nnc(-c2ccc(Cl)cc2Cl)s1] |
| 222620 | >100 | Ryan Scientific (Mount Pleasant, SC) | 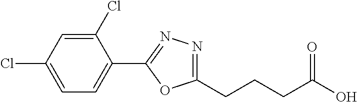 [SMILES: O=C(O)CCCc1nnc(-c2ccc(Cl)cc2Cl)o1] |
| 222623 | 0.0086 | Synthesis Scheme A | 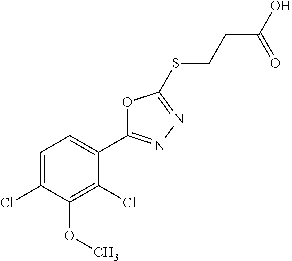 [SMILES: COc1c(Cl)ccc(-c2nnc(SCCC(=O)O)o2)c1Cl] |
| 222687 | 10 | Synthesis Scheme A | 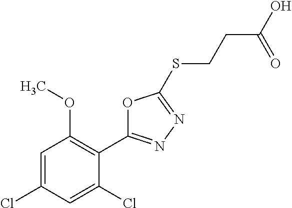 [SMILES: COc1cc(Cl)cc(Cl)c1-c1nnc(SCCC(=O)O)o1] |
| 232001 | 0.0015 | Synthesis Scheme A | 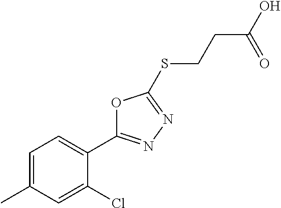 [SMILES: Cc1ccc(-c2nnc(SCCC(=O)O)o2)c(Cl)c1] |
| 232002 | 0.025 | Synthesis Scheme A | 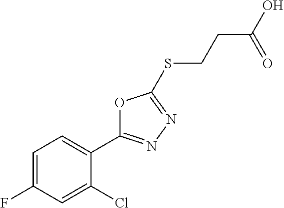 [SMILES: O=C(O)CCSc1nnc(-c2ccc(F)cc2Cl)o1] |
| 232120 | 0.0018 | Synthesis Scheme B | 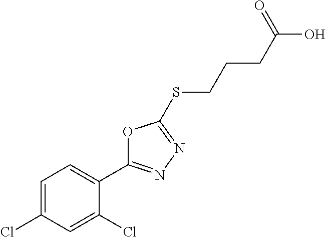 [SMILES: O=C(O)CCCSc1nnc(-c2ccc(Cl)cc2Cl)o1] |
| 232503 | 0.003 | Synthesis Scheme B | 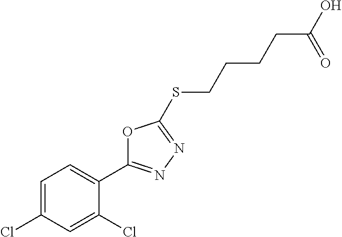 [SMILES: O=C(O)CCCCSc1nnc(-c2ccc(Cl)cc2Cl)o1] |
| 1000 | | Synthesis Scheme B | 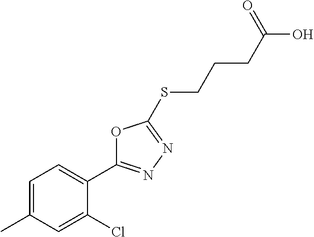 [SMILES: Cc1ccc(-c2nnc(SCCCC(=O)O)o2)c(Cl)c1] |
| 1001 | | Synthesis Scheme B | 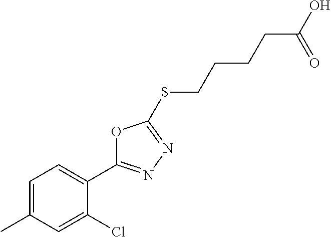 [SMILES: Cc1ccc(-c2nnc(SCCCCC(=O)O)o2)c(Cl)c1] |

### Example 4

### Synthesis Procedures

General Method

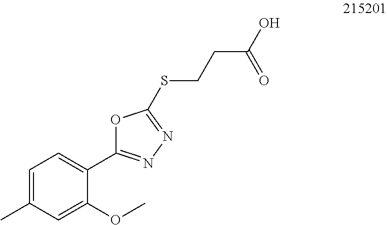 [SMILES: COc1cc(C)ccc1-c1nnc(SCCC(=O)O)o1]

### 2-Methoxy-4-methylbenzohydrazide

To a suspension of 2-methoxy-4-methylbenzoic acid (1.0 g, 6.0 mmol) in methanol (20 ml) was added sulfuric acid (0.10 ml, 1.9 mmol). The reaction was heated to reflux and stirred for 6 h. The reaction was cooled to room temperature and stirred overnight. Hydrazine hydrate (3.0 mL, 60 mmol) was added and the reaction stirred at room temperature for 3 days. The crude reaction mixture was concentrated to 20 mL. The white solid which precipitated out was filtered off. The filtrate was poured into water and extracted with dichloromethane (3×). The combined organic layers were washed with brine, dried over magnesium sulfate, filtered, and concentrated. The resulting solid was combined with the solid which was originally filtered off to give the title compound as a white solid (1.1 g, 99%).

### 5-(2-Methoxy-4-methylphenyl)-1,3,4-oxadiazole-2-thiol

To a solution of 2-methoxy-4-methylbenzohydrazide (870 mg, 4.8 mmol) in ethanol (25 ml) was added a solution of potassium hydroxide (380 mg, 5.8 mmol) in water (1.5 ml, 4.8 mmol). To this mixture was added carbon disulfide (0.29 ml, 4.8 mmol). The clear, colorless solution turned yellow. The reaction mixture was heated to reflux and stirred for 4 h, then left overnight at room temperature. Additional carbon disulfide (100 uL) was added and the reaction stirred at reflux for 3 h. The reaction then stirred at room temperature overnight, then was poured over 20 mL of IN HCl on ice. The white solid which precipitated out was filtered, and washed with cold methanol. The wet solid was dissolved in dichloromethane and washed with water and brine. The organic layer was dried over magnesium sulfate, filtered and concentrated to give the title compound as an off-white solid (1.0 g, 97%).

### tert-Butyl 3-((5-(2-methoxy-4-methylphenyl)-1,3,4-oxadiazol-2-yl)thio)propanoate

To a solution of 5-(2-methoxy-4-methylphenyl)-1,3,4-oxadiazole-2-thiol (380 mg, 1.7 mmol) in acetone (10 ml) was added potassium carbonate (350 mg, 2.6 mmol) followed by tert-butyl 3-bromopropanoate (0.34 ml, 2.0 mmol). The reaction stirred at room temperature overnight. The reaction was poured into dichloromethane and washed with water and brine. The organic layer was dried over magnesium sulfate, filtered and concentrated. The crude residue was purified by column chromatography on silica gel eluting with 5-25% ethyl acetate in hexanes to give the title compound as a white solid (540 mg, 90%).

### 3-((5-(2-Methoxy-4-methylphenyl)-1,3,4-oxadiazol-2-yl)thio)propanoic acid

To a solution of tert-butyl 3-((5-(2-methoxy-4-methylphenyl)-1,3,4-oxadiazol-2-yl)thio)propanoate (250 mg, 0.72 mmol) in dichloromethane (5.0 ml) was added TFA (700 L, 9.1 mmol). The reaction stirred at room temperature overnight. The reaction was poured into water and extracted (2×) with dichloromethane. The combined organic layers were dried over magnesium sulfate, filtered and concentrated. The crude residue was purified by column chromatography eluting with 5-15% methanol in dichloromethane to give the title compound as a white solid (190 mg. 88%). ¹H NMR (400 MHz, DMSO-d6) δ 12.53, 7.67, 7.07, 6.91, 3.85, 3.40, 2.81, 2.37.

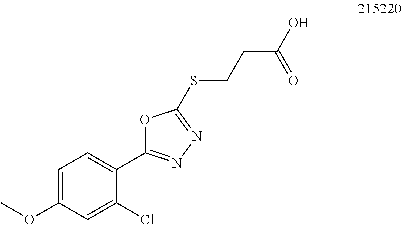 [SMILES: COc1ccc(-c2nnc(SCCC(=O)O)o2)c(Cl)c1]

### 3-((5-(2-Chloro-4-methoxyphenyl)-1,3,4-oxadiazol-2-yl)thio)propanoic acid

Prepared by the General Method from 2-chloro-4-methoxybenzoic acid.

¹H NMR (400 MHz, DMSO-d6) δ 12.55, 7.87, 7.26, 7.10, 3.85, 3.42, 2.81.

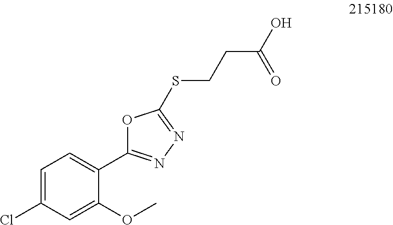 [SMILES: COc1cc(Cl)ccc1-c1nnc(SCCC(=O)O)o1]

### 3-((5-(4-Chloro-2-methoxyphenyl)-1,3,4-oxadiazol-2-yl)thio)propanoic acid

Prepared by the General Method from 4-chloro-2-methoxybenzoic acid.

¹H NMR (400 MHz, DMSO-d6) δ 12.52, 7.81, 7.35, 7.18, 3.90, 3.41, 2.80.

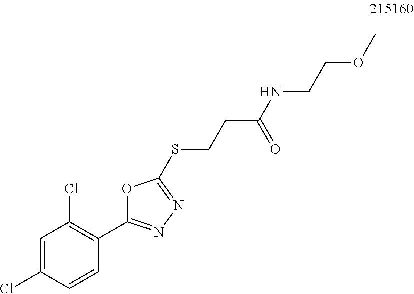 [SMILES: COCCNC(=O)CCSc1nnc(-c2ccc(Cl)cc2Cl)o1]

### 3-((5-(2,4-Dichlorophenyl)-1,3,4-oxadiazol-2-yl)thio)propanoic acid

### Followed General Procedure

### 3-((5-(2,4-Dichlorophenyl)-1,3,4-oxadiazol-2-yl)thio)-N-(2-methoxyethyl)propanamide

To a solution of 3-((5-(2,4-dichlorophenyl)-1,3,4-oxadiazol-2-yl)thio)propanoic acid (100 mg, 0.31 mmol) in tetrahydrofuran (3.0 ml) was added 2-methoxyethan-1-amine (0.054 ml, 0.63 mmol), N-(3-dimethylaminopropyl)-N′-ethylcarbodiimide hydrochloride (90 mg, 0.47 mmol), N,N-diisopropylethylamine (0.11 ml, 0.63 mmol), and 4-(dimethylamino) pyridine (3.8 mg, 0.031 mmol). The reaction stirred overnight at room temperature. The reaction mixture was poured into dichloromethane, washed with saturated sodium bicarbonate solution and brine. The organic layer was dried over magnesium sulfate, filtered, and concentrated. Purified by column chromatography on silica gel eluting with 70-90% ethyl acetate in hexanes. Isolated the title compound as a white solid (89 mg, 75%). ¹H NMR (400 MHz, Chloroform-d) δ 7.90, 7.57, 7.39, 5.95, 3.58, 3.46, 3.35, 2.83.

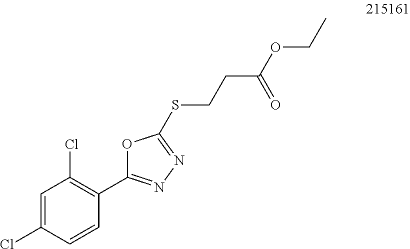 [SMILES: CCOC(=O)CCSc1nnc(-c2ccc(Cl)cc2Cl)o1]

### Ethyl 3-((5-(2,4-dichlorophenyl)-1,3,4-oxadiazol-2-yl)thio)propanoate

### 5-(2,4-Dichlorophenyl)-1,3,4-oxadiazole-2-thiol

### Followed General Procedure

### Ethyl 3-((5-(2,4-dichlorophenyl)-1,3,4-oxadiazol-2-yl)thio)propanoate

To a solution of 5-(2,4-dichlorophenyl)-1,3,4-oxadiazole-2-thiol (1.2 g, 5.1 mmol) in acetone (40 ml) was added potassium carbonate (1.0 g, 7.6 mmol) followed by ethyl 3-bromopropanoate (0.97 ml, 7.6 mmol). The reaction stirred at room temperature for 2 h. Additional ethyl 3-bromopropanoate (500 uL) was added and the reaction stirred at room temperature for another 2 h. The reaction mixture was poured into ethyl acetate and washed with water and brine. The organic layer was dried over magnesium sulfate, filtered and concentrated. Purified by flash chromatography on silica gel eluting with 5-20% ethyl acetate in hexanes to give the title compound as a clear colorless oil (1.6 g, 91%). ¹H NMR (400 MHz, DMSO-d6) δ 7.98, 7.92, 7.65, 4.08, 3.48, 2.90, 1.17.

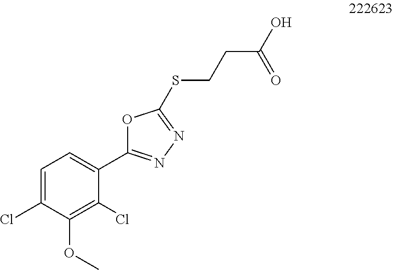 [SMILES: COc1c(Cl)ccc(-c2nnc(SCCC(=O)O)o2)c1Cl]

### 3-((5-(2,4-Dichloro-3-methoxyphenyl)-1,3,4-oxadiazol-2-yl)thio)propanoic acid

### 2,4-Dichloro-3-methoxybenzoic acid

Procedure adapted from Eur. J. Org. Chem. 2006, 4398-4404)

To a solution of 1,3-dichloro-2-methoxybenzene (3.0 ml, 22 mmol) in tetrahydrofuran (75 ml) at −78° C. was added sec-butyllithium (1.4M in cyclohexanes) (17 ml, 24 mmol). The reaction turned from colorless to pale yellow. The reaction stirred at −78° C. for 45 minutes, at which time freshly crushed dry ice was added. Vigorous gas evolution occurred. The dry ice bath was removed and the reaction was allowed to warm for 30 minutes. The reaction mixture (which was still cold) was poured onto IN HCl and extracted with ethyl acetate (3×). The combined organic layers were dried over magnesium sulfate, filtered, and concentrated. The white solid was dissolved in dichloromethane and hexanes was added until cloudy. The solid which had precipitated out was filtered off, was washed with hexanes and dried under vacuum to give the title compound as a white solid (1.2 g, 24%).

### 3-((5-(2,4-Dichloro-3-methoxyphenyl)-1,3,4-oxadiazol-2-yl)thio)propanoic acid

Prepared by the General Method from 2,4-dichloro-3-methoxybenzoic acid.

¹H NMR (400 MHz, Chloroform-d) δ 12.54, 7.73, 7.71, 3.87, 3.44, 2.82.

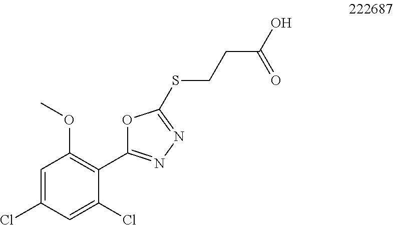 [SMILES: COc1cc(Cl)cc(Cl)c1-c1nnc(SCCC(=O)O)o1]

### 3-((5-(2,4-Dichloro-6-methoxyphenyl)-1,3,4-oxadiazol-2-yl)thio)propanoic acid

### 2,4-Dichloro-6-methoxybenzoic acid

Procedure followed J. Org. Chem. 1985, 50, 408-410

To solution of nBuLi (2.5 M in hexanes) (8.8 ml, 22 mmol) in tetrahydrofuran (20 ml) at −78° C. was added N,N,N′,N′-tetramethylethylenediamine (3.6 ml, 23 mmol) and the solution was stirred for 30 minutes at −78° C. A solution of 1,3-dichloro-5-methoxybenzene (3.0 g, 17 mmol) in tetrahydrofuran (7.0 mL) was added slowly, and the reaction mixture was stirred for an additional 1.5 h. Crushed solid carbon dioxide (CO₂) was added portionwise, and the reaction warmed to room temperature over 2 h. Kept adding crushed CO₂ over this time period. The reaction mixture was then poured into IN HCl, and extracted 3× with dichloromethane. The combined organic layers were dried over magnesium sulfate, and filtered. The solution was poured into dichloromethane and basified with IN aqueous sodium hydroxide solution. The aqueous layer was washed with dichloromethane, and then acidified with IN aqueous HCl solution. This solution was extracted 3× with dichloromethane. The combined organic extracts were dried over magnesium sulfate, filtered and concentrated to give 2.2 g of a pale green solid as a mixture of regioisomers. The mixture was taken into the next step without purification.

### Methyl 2,4-dichloro-6-methoxybenzoate

Procedure followed J. Org. Chem. 1985, 50, 408-410

A solution of 2,4-dichloro-6-methoxybenzoic acid with 2,6-dichloro-4-methoxybenzoic acid (1:1) (2.2 g, 4.9 mmol) in thionyl chloride (30 ml, 410 mmol) was heated to reflux and stirred for 1.5 h. The reaction was cooled to room temperature and stirred overnight. The reaction was concentrated on rotovap to remove thionyl chloride. Methanol (40 ml) and pyridine (0.80 ml, 9.9 mmol) were added and the reaction stirred at room temperature over the weekend. The reaction was poured into IN HCl and extracted 3× with dichloromethane. The combined organic layers were dried over magnesium sulfate, filtered and concentrated. The crude reaction mixture was purified by column chromatography on silica gel eluting with 2-10% ethyl acetate in hexanes to give methyl 2,6-dichloro-4-methoxybenzoate (270 mg, 23%) and the title compound as a colorless oil which slowly solidified. (780 mg, 67%).

### 3-((5-(2,4-Dichloro-6-methoxyphenyl)-1,3,4-oxadiazol-2-yl)thio)propanoic acid

Prepared by the General Method from 2,4-chloro-6-methoxybenzoic acid.

¹H NMR (400 MHz, DMSO-d6) δ 12.60, 7.46, 7.38, 3.84, 3.43, 2.80.

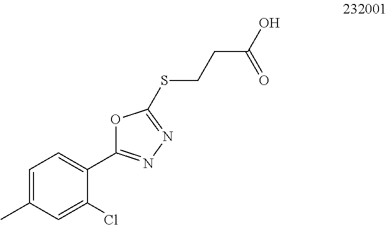 [SMILES: Cc1ccc(-c2nnc(SCCC(=O)O)o2)c(Cl)c1]

### 3-((5-(2-chloro-4-methylphenyl)-1,3,4-oxadiazol-2-yl)thio)propanoic acid

Prepared by the General Method from 2-chloro-4-methylbenzoic acid.

¹H NMR (400 MHz, DMSO-d₆) δ 12.47, 7.85, 7.55, 7.37, 3.46, 2.84, 2.40.

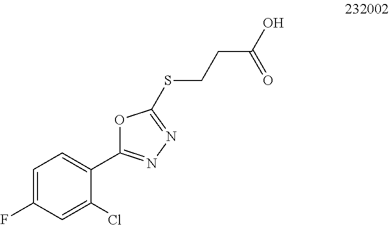 [SMILES: O=C(O)CCSc1nnc(-c2ccc(F)cc2Cl)o1]

### 3-((5-(2-chloro-4-fluorophenyl)-1,3,4-oxadiazol-2-yl)thio)propanoic acid

Prepared by the General Method from 2-chloro-4-fluorobenzoic acid.

¹H NMR (400 MHz, DMSO-d₆) δ 12.56, 8.05, 7.76, 7.48, 3.46, 2.84.

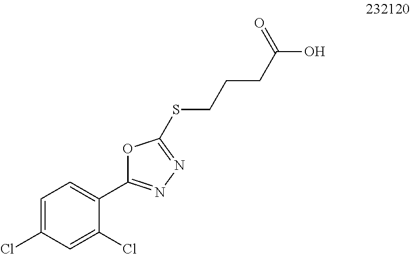 [SMILES: O=C(O)CCCSc1nnc(-c2ccc(Cl)cc2Cl)o1]

### 4-((5-(2,4-dichlorophenyl)-1,3,4-oxadiazol-2-yl)thio)butanoic acid

Prepared by a method analogous to that shown in Scheme B.

### methyl-4-((5-(2,4-dichlorophenyl)-1,3,4-oxadiazol-2-yl)thio)butanoate

To a solution of 5-(2,4-dichlorophenyl)-1,3,4-oxadiazole-2-thiol (200 mg, 0.80 mmol) in acetone (10 mL), was added potassium carbonate (130 mg, 0.97 mmol) followed methyl 4-bromobutanoate (0.12 mL, 0.97 mmol) and the reaction was stirred under nitrogen at 25° C. for 4 h. The crude mixture was concentrated to remove the acetone, and the residue was partitioned between dichloromethane and water. The aqueous layer was extracted with dichloromethane and the combined organic layers were washed with brine, dried over magnesium sulfate, filtered and concentrated to a tan oil. The crude residue was purified by column chromatography eluting with 20% ethyl acetate in hexanes to give the title compound as an oil which solidified upon standing (190 mg, 66%). ¹H NMR (400 MHz, DMSO-d₆) δ ppm 8.00, 7.92, 7.66, 3.60, 3.35, 2.52, 2.07.

### 4-((5-(2,4-dichlorophenyl)-1,3,4-oxadiazol-2-yl)thio)butanoic acid

To a solution of methyl-4-((5-(2,4-dichlorophenyl)-1,3,4-oxadiazol-2-yl)thio)butanoate (0.19 g, 0.54 mmol) in tetrahydrofuran (3 mL) was added 1 M NaOH (3 mL) and the reaction was stirred at 25° C. for 16 h. The tetrahydrofuran was concentrated and the aqueous solution was washed with dichloromethane (3×10 mL). The aqueous layer was acidified with 1 N HCl (7 mL) and the product was extracted with ethyl acetate (3×10 mL). The combined organic extracts were washed with brine (3×10 mL), dried over magnesium sulfate, filtered, and concentrated. The crude residue was dissolved in ethyl acetate (2 mL) and the product was precipitated with hexanes (5 mL). The solid was filtered and dried under vacuum to give the title compound as tan solid (63 mg, 35%). ¹H NMR (400 MHz, DMSO-d₆) δ 12.21, 8.00, 7.93, 7.66, 3.34, 2.42, 2.01.

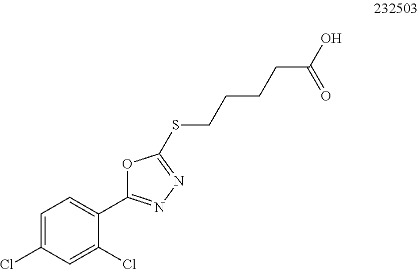 [SMILES: O=C(O)CCCCSc1nnc(-c2ccc(Cl)cc2Cl)o1]

### 5-((5-(2,4-dichlorophenyl)-1,3,4-oxadiazol-2-yl)thio)pentanoic acid

Prepared by a method analogous to that shown in Scheme B.

### methyl 5-((5-(2,4-dichlorophenyl)-1,3,4-oxadiazol-2-yl)thio)pentanoate

To a solution of 5-(2,4-dichlorophenyl)-1,3,4-oxadiazole-2-thiol (275 mg, 1.11 mmol) in acetone (10 mL), was added potassium carbonate (230 mg, 1.67 mmol) followed methyl 5-bromopentanoate (330 mg, 1.67 mmol). The reaction was stirred under nitrogen at 25° C. for 6 h. The crude mixture was poured into water and extracted with dichloromethane. The organic layer was washed with brine, dried over magnesium sulfate, filtered and concentrated. The crude residue was purified by column chromatography eluting with 10-30% ethyl acetate in hexanes to give the title compound as an oil which solidified upon standing (330 mg, 82%). ¹H NMR (400 MHz, DMSO-d₆) δ ppm 7.97, 7.90, 7.64, 3.55, 3.30, 2.35, 1.77, 1.65.

### 5-((5-(2,4-dichlorophenyl)-1,3,4-oxadiazol-2-yl)thio)pentanoic acid

To a solution of methyl-5-((5-(2,4-dichlorophenyl)-1,3,4-oxadiazol-2-yl)thio)pentanoate (330 mg, 0.91 mmol) in tetrahydrofuran (5 mL) was added 1 M NaOH (5 mL). The reaction was stirred at 25° C. for 16 h. The tetrahydrofuran was concentrated and the aqueous solution was washed with dichloromethane (10 mL). The aqueous layer was acidified with 1 N HCl (7 mL) and the product was extracted with dichloromethane (3×10 mL). The combined organic extracts were washed with brine (3×10 mL), dried over magnesium sulfate, filtered, and concentrated. The crude residue was purified by chromatography eluting with 50% ethyl acetate in hexanes then with 2-10% methanol in dichloromethane to give the title compound as a white solid (110 mg, 34%). ¹H NMR (400 MHz, DMSO-d₆) δ ppm 12.05, 7.97, 7.90, 7.64, 3.30, 2.25, 1.78, 1.62.

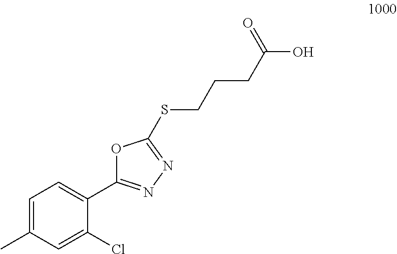 [SMILES: Cc1ccc(-c2nnc(SCCCC(=O)O)o2)c(Cl)c1]

### 4-((5-(2chloro-4-methylphenyl)-1,3,4-oxadiazol-2-yl)thio)butanoic acid

Compound 1000 is prepared by a method analogous to that described for 232120 starting with 2-chloro-4-methylbenzoic acid instead of 2,4-dichlorobenzoic acid.

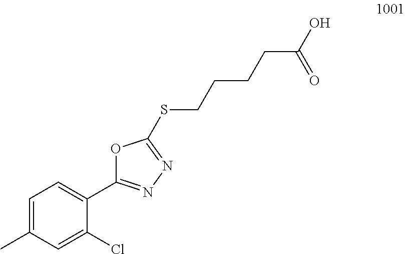 [SMILES: Cc1ccc(-c2nnc(SCCCCC(=O)O)o2)c(Cl)c1]

### 5-((5-(2-chloro-4-methylphenyl)-1,3,4-oxadiazol-2-yl)thio)pentanoic acid

Compound 1001 is prepared by a method analogous to that described for 232503 starting with 2-chloro-4-methylbenzoic acid instead of 2,4-dichlorobenzoic acid.

All publications and patents mentioned in the above specification are herein incorporated by reference. Various modifications and variations of the described method and system of the disclosure will be apparent to those skilled in the art without departing from the scope and spirit of the disclosure. Although the disclosure has been described in connection with specific preferred embodiments, it should be understood that the disclosure as claimed should not be unduly limited to such specific embodiments. Indeed, various modifications of the described modes for carrying out the disclosure that are obvious to those skilled in molecular biology, cancer biology, genetics, or related fields are intended to be within the scope of the following claims.

We claim:

1. A method of treating or preventing a rho-mediated disease in a subject comprising administering a compound of structure I, 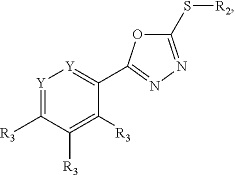
wherein Y is C—R3 or N, R2 is (CH2)nCOOR1, wherein each CH2 group may be optionally substituted, R1 is H or C1-C6 alkyl, n is an integer from 1 to 10, and R3 is the same or different and is H, a halide, an ether, or a straight or branched alkyl.

2. The method of claim 1, wherein said composition is selected from the group consisting of

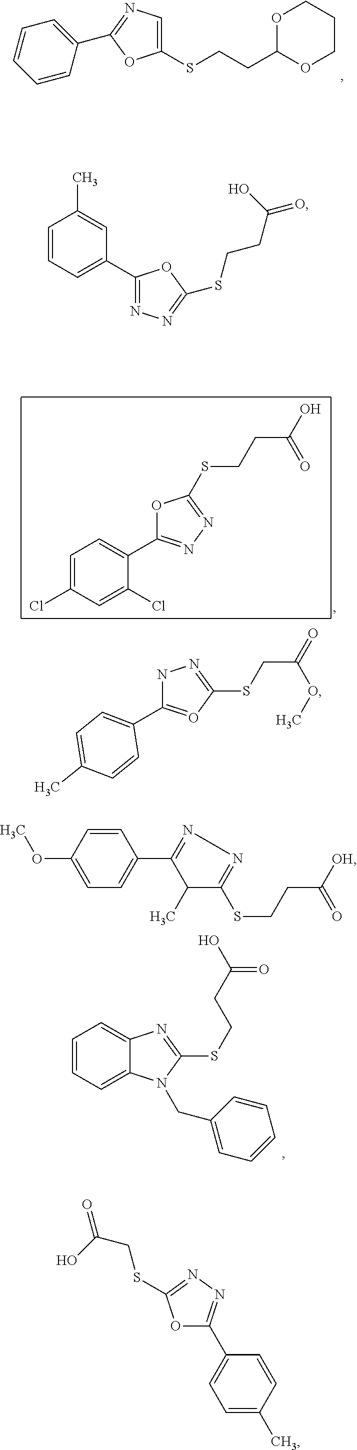

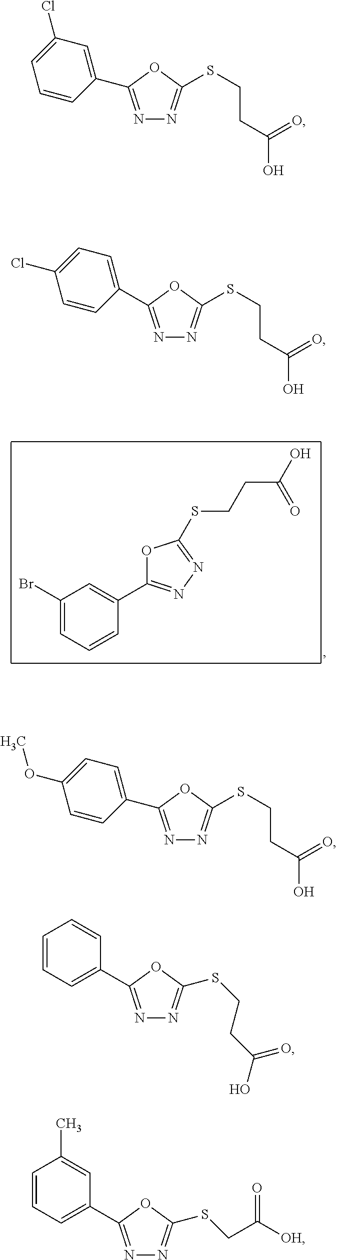 [SMILES: O=C(O)CCSc1nnc(-c2cccc(Cl)c2)o1 ; O=C(O)CCSc1nnc(-c2ccc(Cl)cc2)o1 ; O=C(O)CCSc1nnc(-c2cccc(Br)c2)o1 ; COc1ccc(-c2nnc(SCCC(=O)O)o2)cc1 ; O=C(O)CCSc1nnc(-c2ccccc2)o1 ; Cc1cccc(-c2nnc(SCC(=O)O)o2)c1]

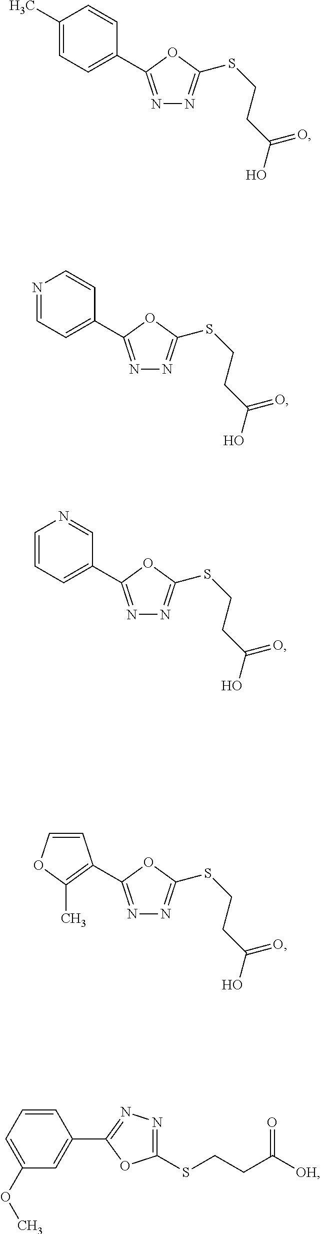 [SMILES: Cc1ccc(-c2nnc(SCCC(=O)O)o2)cc1 ; O=C(O)CCSc1nnc(-c2ccncc2)o1 ; O=C(O)CCSc1nnc(-c2cccnc2)o1 ; Cc1occc1-c1nnc(SCCC(=O)O)o1 ; COc1cccc(-c2nnc(SCCC(=O)O)o2)c1]

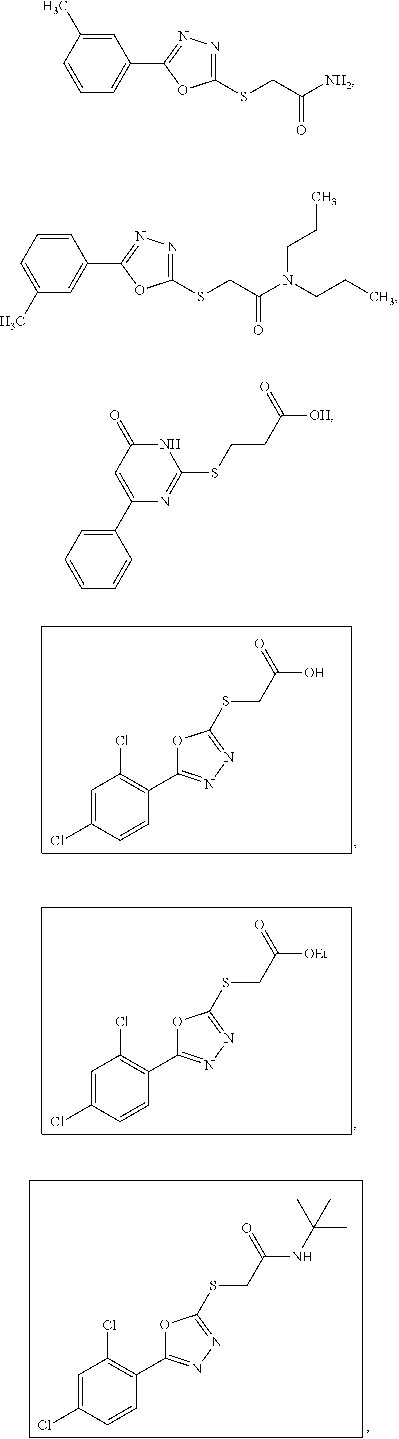 [SMILES: Cc1cccc(-c2nnc(SCC(N)=O)o2)c1 ; CCCN(CCC)C(=O)CSc1nnc(-c2cccc(C)c2)o1 ; O=C(O)CCSc1nc(-c2ccccc2)cc(=O)[nH]1 ; O=C(O)CSc1nnc(-c2ccc(Cl)cc2Cl)o1 ; CCOC(=O)CSc1nnc(-c2ccc(Cl)cc2Cl)o1 ; CC(C)(C)NC(=O)CSc1nnc(-c2ccc(Cl)cc2Cl)o1]

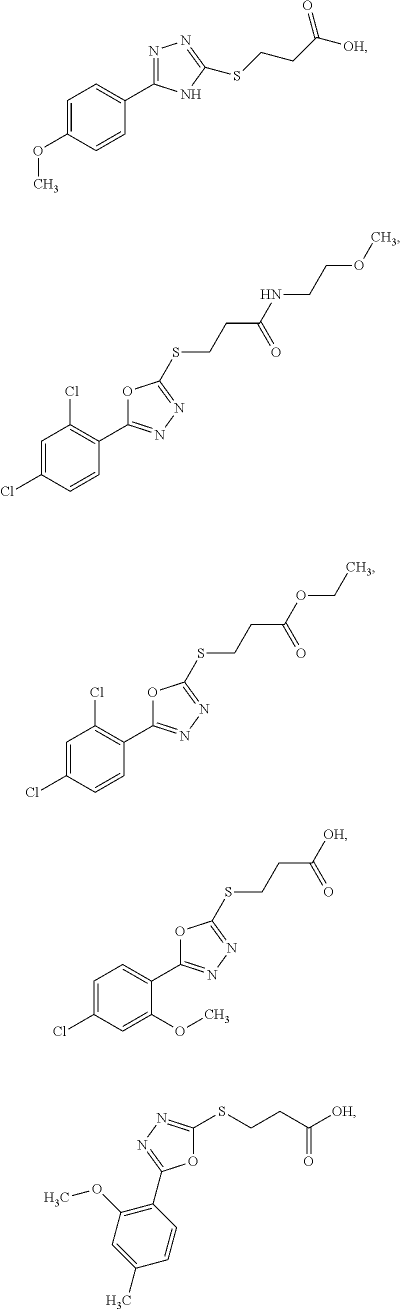 [SMILES: COc1ccc(-c2nnc(SCCC(=O)O)[nH]2)cc1 ; COCCNC(=O)CCSc1nnc(-c2ccc(Cl)cc2Cl)o1 ; CCOC(=O)CCSc1nnc(-c2ccc(Cl)cc2Cl)o1 ; COc1cc(Cl)ccc1-c1nnc(SCCC(=O)O)o1 ; COc1cc(C)ccc1-c1nnc(SCCC(=O)O)o1]

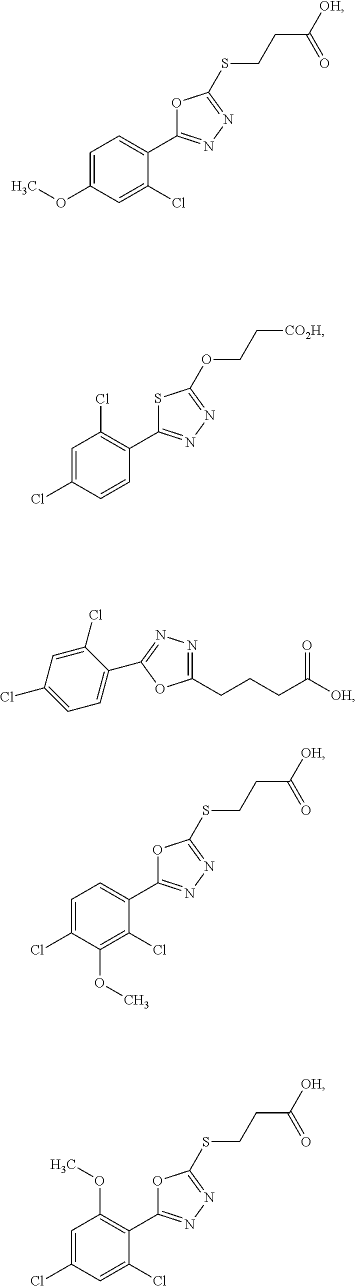 [SMILES: COc1ccc(-c2nnc(SCCC(=O)O)o2)c(Cl)c1 ; O=C(O)CCOc1nnc(-c2ccc(Cl)cc2Cl)s1 ; O=C(O)CCCc1nnc(-c2ccc(Cl)cc2Cl)o1 ; COc1c(Cl)ccc(-c2nnc(SCCC(=O)O)o2)c1Cl ; COc1cc(Cl)cc(Cl)c1-c1nnc(SCCC(=O)O)o1]

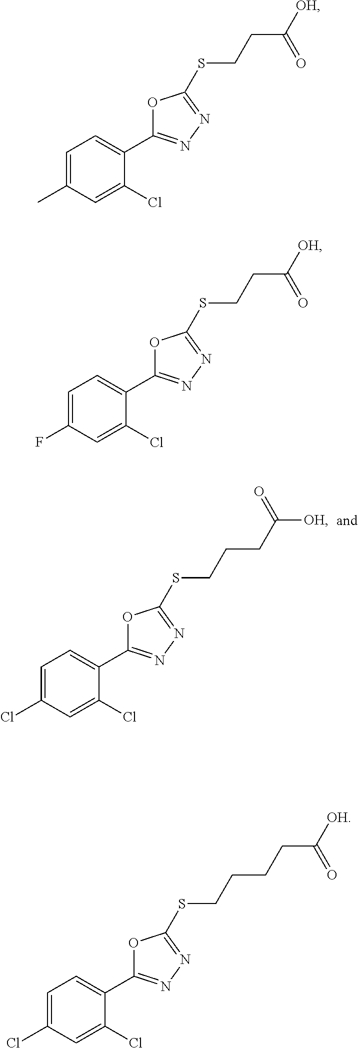 [SMILES: Cc1ccc(-c2nnc(SCCC(=O)O)o2)c(Cl)c1 ; O=C(O)CCSc1nnc(-c2ccc(F)cc2Cl)o1 ; O=C(O)CCCSc1nnc(-c2ccc(Cl)cc2Cl)o1 ; O=C(O)CCCCSc1nnc(-c2ccc(Cl)cc2Cl)o1]

3. The method of claim 1, wherein said rho-mediated disease is selected from the group consisting of fibrosis, cancer, inflammation, inflammatory disease, pulmonary arterial hypertension, axon regeneration following nerve damage, Raynaud's phenomenon, cerebral vascular disease, cardiovascular disease, and erectile dysfunction.

4. The method of claim 3, wherein said fibrosis is systemic sclerosis.

5. The method of claim 3, wherein said inflammatory disease is selected from the group consisting of Crohn's Disease, idiopathic pulmonary fibrosis, arthritis, rheumatoid arthritis, psoriatic arthritis, osteoarthritis, degenerative arthritis, polymyalgia rheumatic, ankylosing spondylitis, reactive arthritis, gout, pseudogout, inflammatory joint disease, systemic lupus erythematosus, polymyositis, fibromyalgis, achilles tendinitis, achondroplasia, acromegalic arthropathy, adhesive capsulitis, adult onset Still's disease, anserine bursitis, avascular necrosis, Behcet's syndrome, bicipital tendinitis, Blount's disease, brucellar spondylitis, bursitis, calcaneal bursitis, calcium pyrophosphate deposition disease, crystal deposition disease, Caplan's syndrome, carpal tunnel syndrome, chondrocalcinosis, chondromalacia patellae, chronic synovitis, chronic recurrent multifocal osteomyelitis, Churg-Strauss syndrome, Cogan's syndrome, corticosteroid-induced osteoporosis, costosternal syndrome, CREST syndrome, cryoglobulinemia, degenerative joint disease, dermatomyositis, diabetic finger sclerosis, diffuse idiopathic skeletal hyperostosis, discitis, discoid lupus erythematosus, drug-induced lupus, Duchenne's muscular dystrophy, Dupuytren's contracture, Ehlers-Danlos syndrome, enteropathic arthritis, epicondylitis, erosive inflammatory osteoarthritis, exercise-induced compartment syndrome, Fabry's disease, familial Mediterranean fever, Farber's lipogranulomatosis, Felty's syndrome, Fifth's disease, flat feet, foreign body synovitis, Freiberg's disease, fungal arthritis, Gaucher's disease, giant cell arteritis, gonococcal arthritis, Goodpasture's syndrome, granulomatous arteritis, hemarthrosis, hemochromatosis, Henoch-Schonlein purpura, Hepatitis B surface antigen disease, hip dysplasia, Hurler syndrome, hypermobility syndrome, hypersensitivity vasculitis, hypertrophic osteoarthropathy, immune complex disease, impingement syndrome, Jaccoud's arthropathy, juvenile ankylosing spondylitis, juvenile dermatomyositis, juvenile rheumatoid arthritis, Kawasaki disease, Kienbock's disease, Legg-Calve-Perthes disease, Lesch-Nyhan syndrome, linear scleroderma, lipoid dermatoarthritis, Lofgren's syndrome, Lyme disease, malignant synovioma, Marfan's syndrome, medial plica syndrome, metastatic carcinomatous arthritis, mixed connective tissue disease, mixed cryoglobulinemia, mucopolysaccharidosis, multicentric reticulohistiocytosis, multiple epiphyseal dysplasia, mycoplasmal arthritis, myofascial pain syndrome, neonatal lupus, neuropathic arthropathy, nodular panniculitis, ochronosis, olecranon bursitis, Osgood-Schlatter's disease, osteoarthritis, osteochondromatosis, osteogenesis imperfecta, osteomalacia, osteomyelitis, osteonecrosis, osteoporosis, overlap syndrome, pachydermoperiostosis Paget's disease of bone, palindromic rheumatism, patellofemoral pain syndrome, Pellegrini-Stieda syndrome, pigmented villonodular synovitis, piriformis syndrome, plantar fasciitis, polyarteritis nodos, Polymyalgia rheumatic, polymyositis, popliteal cysts, posterior tibial tendinitis, Pott's disease, prepatellar bursitis, prosthetic joint infection, pseudoxanthoma elasticum, psoriatic arthritis, Raynaud's phenomenon, reactive arthritis/Reiter's syndrome, reflex sympathetic dystrophy syndrome, relapsing polychondritis, retrocalcaneal bursitis, rheumatic fever, rheumatoid vasculitis, rotator cuff tendinitis, sacroiliitis, salmonella osteomyelitis, sarcoidosis, saturnine gout, Scheuermann's osteochondritis, scleroderma, septic arthritis, seronegative arthritis, shigella arthritis, shoulder-hand syndrome, sickle cell arthropathy, Sjogren's syndrome, slipped capital femoral epiphysis, spinal stenosis, spondylolysis, staphylococcus arthritis, Stickler syndrome, subacute cutaneous lupus, Sweet's syndrome, Sydenham's chorea, syphilitic arthritis, systemic lupus erythematosus, Takayasu's arteritis, tarsal tunnel syndrome, tennis elbow, Tietse's syndrome, transient osteoporosis, traumatic arthritis, trochanteric bursitis, tuberculosis arthritis, arthritis of Ulcerative colitis, undifferentiated connective tissue syndrome, urticarial vasculitis, viral arthritis, Wegener's granulomatosis, Whipple's disease, Wilson's disease, and yersinial arthritis.

6. The method of claim 1, further comprising administering an agent selected from the group consisting of a chemotherapeutic agent, an anti-fibrotic agent, and an anti-inflammatory agent.

7. A pharmaceutical composition comprising a compound of the structure: 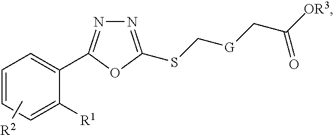
wherein R1 is halogen, or a C1-C5 straight or branched chain alkyl, C1-C3 alkyl-O; R2 is H, halogen, a C1-C5 straight or branched chain alkyl, or C1-C3 alkyl-O; R3 is H or C1-C3 alkyl; and G is (CH₂)ₙ wherein n=1 or 2.

8. The composition of claim 7, wherein when n=1 and R1 is Me, R2 is not 4-Me or H, and when n=1 and R1 is OMe, R2 is not H.

9. The composition of claim 8, wherein said composition is selected from the group consisting of

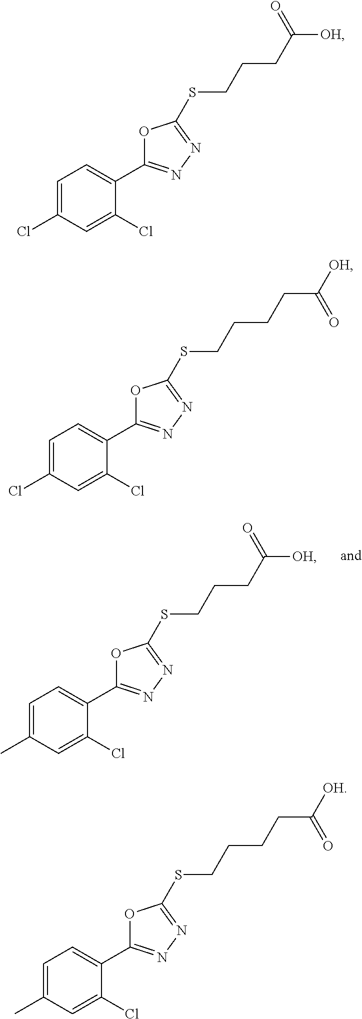 [SMILES: O=C(O)CCCSc1nnc(-c2ccc(Cl)cc2Cl)o1 ; O=C(O)CCCCSc1nnc(-c2ccc(Cl)cc2Cl)o1 ; Cc1ccc(-c2nnc(SCCCC(=O)O)o2)c(Cl)c1 ; Cc1ccc(-c2nnc(SCCCCC(=O)O)o2)c(Cl)c1]

10. The composition of claim 7, wherein said composition is in a pharmaceutically appropriate formulation for administration to a human subject.

11. The composition of claim 7, wherein said composition comprises a pharmaceutically acceptable carrier.

12. The composition of claim 7, wherein said composition further comprises an additional agent selected from the group consisting of a chemotherapeutic agent, an anti-fibrotic agent, and an anti-inflammatory agent.

13. A composition, comprising a compound selected from

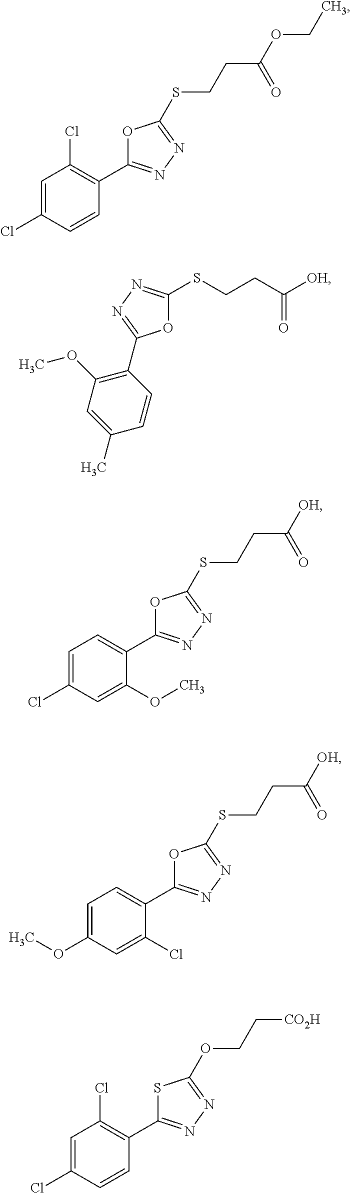 [SMILES: CCOC(=O)CCSc1nnc(-c2ccc(Cl)cc2Cl)o1 ; COc1cc(C)ccc1-c1nnc(SCCC(=O)O)o1 ; COc1cc(Cl)ccc1-c1nnc(SCCC(=O)O)o1 ; COc1ccc(-c2nnc(SCCC(=O)O)o2)c(Cl)c1 ; O=C(O)CCOc1nnc(-c2ccc(Cl)cc2Cl)s1]

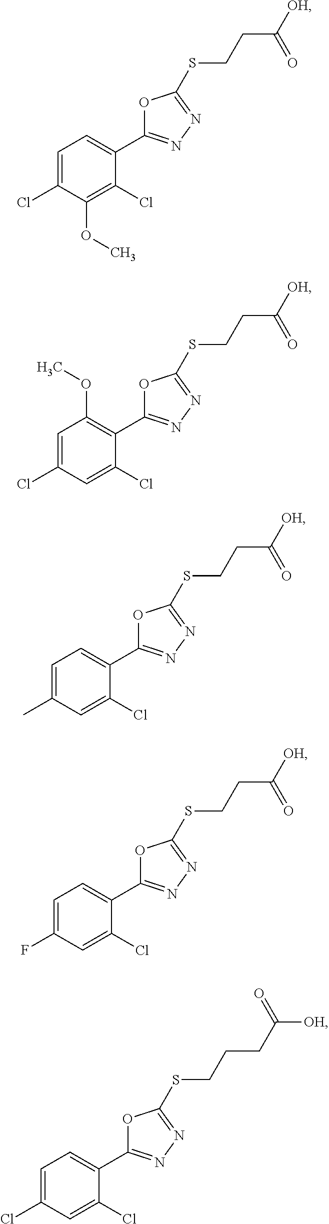 [SMILES: COc1c(Cl)ccc(-c2nnc(SCCC(=O)O)o2)c1Cl ; COc1cc(Cl)cc(Cl)c1-c1nnc(SCCC(=O)O)o1 ; Cc1ccc(-c2nnc(SCCC(=O)O)o2)c(Cl)c1 ; O=C(O)CCSc1nnc(-c2ccc(F)cc2Cl)o1 ; O=C(O)CCCSc1nnc(-c2ccc(Cl)cc2Cl)o1]

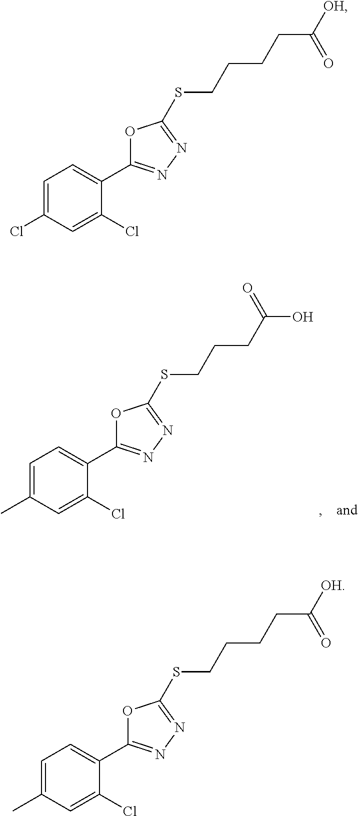 [SMILES: O=C(O)CCCCSc1nnc(-c2ccc(Cl)cc2Cl)o1 ; Cc1ccc(-c2nnc(SCCCC(=O)O)o2)c(Cl)c1 ; Cc1ccc(-c2nnc(SCCCCC(=O)O)o2)c(Cl)c1]

14. The composition of claim 13, wherein said composition is in a pharmaceutically appropriate formulation for administration to a human subject.

15. The composition of claim 14, wherein said composition comprises a pharmaceutically acceptable carrier.

16. The composition of claim 15, wherein said composition further comprises an additional agent selected from the group consisting of a chemotherapeutic agent, an anti-fibrotic agent, and an anti-inflammatory agent.
